(19) United States
(12) Patent Application Publication
(10) Pub. No.: US 2026/0033614 A1
(43) Pub. Date: Feb. 5, 2026

(54) COSMETIC LAMINATE, COVER COAT LAYER, NAIL PRODUCTION METHOD, AND NAIL REMOVAL METHOD

(71) Applicant: Panasonic Intellectual Property Management Co., Ltd., Osaka (JP)

(72) Inventors: TOMOKO KAWASHIMA, Osaka (JP); HARUKA KUSUKAME, Nara (JP)

(21) Appl. No.: 18/996,275

(22) PCT Filed: Jul. 27, 2023
(86) PCT No.: PCT/JP2023/027517

§ 371 Date: Jan. 17, 2025

(30) Foreign Application Priority Data
Aug. 1, 2022 (JP) 2022-123021

(51) Int. Cl. A45D 31/00; A41G 5/02; A61K 8/86; A61Q 3/02; A61Q 3/04

(52) U.S. Cl. CPC A45D 31/00; A61K 8/86; A61Q 3/02; A61Q 3/04; A41G 5/02

(57) ABSTRACT

The present disclosure provides a cosmetic laminate that can firmly adhere to a part of the living body and can be easily peeled off from the living body at any timing. The cosmetic laminate of the present disclosure includes a cosmetic layer adhering to a part of living body and a cover coat layer. The cosmetic layer and the cover coat layer are disposed, the cosmetic layer being positioned between the living body and the cover coat layer. The cosmetic layer contains a resin composition for a cosmetic including a stimulus-responsive material that undergoes a volume change by an external stimulus, and adheres to a part of the living body with a solidified matter of the resin composition for a cosmetic. A water contact angle with respect to a surface of the solidified matter of the resin composition for a cosmetic is from 30° to 110°, inclusive. The cosmetic layer reduces an adhesion strength with a part of the living body by a volume change of the solidified matter caused by application of the external stimulus to the solidified matter of the resin composition for a cosmetic. The cover coat layer has a porosity of less than or equal to 40%.

### TECHNICAL FIELD

The present disclosure relates to a cosmetic laminate, a cover coat layer, a nail production method, and a nail removal method.

### BACKGROUND ART

In recent years, many people use cosmetics that can be continuously worn on a living body for a period of several days to several weeks, such as gel nails and eyelash extensions. In a conventional technique, for example, a cosmetic for a nail such as the gel nail firmly adheres to an own nail by using a photocurable resin or the like. Furthermore, a lash cosmetic such as the eyelash extension firmly adheres to eyelashes by using a humidity curable resin or the like. When these cosmetics are peeled off from the living body, it is common to use a solvent such as acetone. When the cosmetic for a nail is peeled off from the living body, polishing is often performed before or after the solvent is used.

PTL 1 discloses an artificial nail composition that allows an artificial nail to firmly adhere to the own nail by using radical polymerization.

PTL 2 discloses an artificial nail raw material composition that includes a radical polymerizable compound, a polyfunctional thiol compound having a thiol group, and a photopolymerization initiator. By using the artificial nail raw material composition, an adhesive property of an artificial nail to the own nail can be maintained for a long period without using an adhesive and a primer. When the artificial nail obtained by curing the artificial nail raw material composition is removed, a method is used to firstly scrap and scratch the artificial nail, then to dispose cotton immersed in acetone on the artificial nail for about 10 minutes, and then to peel off the artificial nail from the own nail by using, for example, a stick.

PTL 3 discloses an artificial nail composition that includes an ionic monomer polymerizable by irradiation with ultraviolet rays. An artificial nail obtained by curing the artificial nail composition can be removed from the own nail with an acid having a pH of less than or equal to 3.5.

PTL 4 discloses a method and a kit for treating a nail surface, capable of removing a cover (for example, a gel nail or an artificial nail) applied to the nail surface by solvent immersion for less than 20 minutes, peeling, or a combination thereof.

As an adhesive for an eyelash extension, for example, cyanoacrylate is generally used. The cyanoacrylate is an adhesive having high adhesive force, and is cured to adhere by polymerization in accordance with humidity, for example, in air. The cyanoacrylate is called an instant adhesive and widely used not only in industrial and medical applications but also in general households. As for the eyelash extension or the like, artificial lashes can adhere to the eyelashes for a period of several weeks by allowing the lashes to firmly adhere to the artificial lashes by using a cyanoacrylate adhesive. PTL 5 discloses a cyanoacrylate-containing composition that includes cyanoacrylate and fullerene. The cyanoacrylate-containing composition can be used as an adhesive composition used for eyelash extensions or the like, and is also excellent in storage stability.

### CITATION LIST

### Patent Literature

PTL 1: Unexamined Japanese Patent Publication No. 2010-53097
PTL 2: Unexamined Japanese Patent Publication No. 2014-5260
PTL 3: Unexamined Japanese Patent Publication No. 2009-5260
PTL 4: Japanese Translation of PCT International Application No. 2019-512000
PTL 5: Unexamined Japanese Patent Publication No. 2016-121299

### SUMMARY OF THE INVENTION

### Technical Problem

Resin compositions for cosmetics, such as a photocurable resin used in gel nails and cyanoacrylate as a humidity curable resin used in eyelash extensions can adhere to a living body such as nails and lashes with high strength. However, in order to peel off the resin compositions for cosmetics from the living body, it is necessary to perform polishing or to use a solvent such as acetone, an acid, or the like, and it has been a large burden for the living body such as the nails and the lashes to be subjected to peeling treatment. On the other hand, in a case where the adhesive property of the resin composition for a cosmetic to the living body is reduced in order to facilitate the peeling treatment, there arises a problem that the cosmetic cannot be continuously worn on the living body for a desired period.

The present disclosure provides a cosmetic laminate that can firmly adhere to a part of the living body and can be easily peeled off from the living body at any timing.

### Solution to Problem

The present disclosure provides a cosmetic laminate including:
a cosmetic layer adhering to a part of a living body; and
a cover coat layer,
in which the cosmetic layer and the cover coat layer are disposed, the cosmetic layer being positioned between the living body and the cover coat layer in a state where the cosmetic laminate is adhered to the living body,
the cosmetic layer contains a resin composition for a cosmetic including a stimulus-responsive material that undergoes a volume change by an external stimulus (provided that, an external stimulus due to contact with a solvent and an external stimulus due to contact with a solution are excluded), and adheres to the part of the living body with a solidified matter of the resin composition for a cosmetic,
a water contact angle with respect to a surface of the solidified matter of the resin composition for a cosmetic is from 30° to 110°, inclusive,
the cosmetic layer reduces an adhesion strength with the part of the living body by a volume change of the solidified matter caused by application of the external stimulus to the solidified matter of the resin composition for a cosmetic, and
the cover coat layer has a porosity of less than or equal to 40%.

In another aspect, the present disclosure provides a cosmetic laminate including:
a cosmetic layer adhering to a part of a living body; and
a cover coat layer,
in which the cosmetic layer and the cover coat layer are disposed, the cosmetic layer being positioned between the living body and the cover coat layer in a state where the cosmetic laminate is adhered to the living body,
the cosmetic layer contains a resin composition for a cosmetic including a stimulus-responsive material that undergoes a volume change due to generation of a gas by an external stimulus (provided that, an external stimulus due to contact with a solvent and an external stimulus due to contact with a solution are excluded), and adheres to the part of the living body with a solidified matter of the resin composition for a cosmetic,
the cosmetic layer reduces an adhesion strength with the part of the living body by a volume change of the solidified matter caused by application of the external stimulus to the solidified matter of the resin composition for a cosmetic, and
the cover coat layer has a gas permeability of less than or equal to 1.5×10⁸ cc/m²·24 hr·atm.

In another aspect, the present disclosure provides a cover coat layer covering at least a part of a cosmetic layer that undergoes a volume change by an external stimulus (provided that, an external stimulus due to contact with a solvent and an external stimulus due to contact with a solution are excluded),
the cover coat layer having a porosity of less than or equal to 40% and a tensile modulus of more than or equal to 5 MPa.

In another aspect, the present disclosure provides a nail production method including:
applying a resin composition for a nail including a stimulus-responsive material that undergoes a volume change by an external stimulus (provided that, an external stimulus due to contact with a solvent and an external stimulus due to contact with a solution are excluded) to a fingernail, and solidifying the resin composition for a nail to form a first nail layer adhered to the fingernail; and
forming a cover coat layer having a porosity of less than or equal to 40% on the first nail layer.

In another aspect, the present disclosure provides a method of removing a nail adhered to a fingernail, the nail including a first nail layer formed of a solidified resin composition for a nail including a stimulus-responsive material that undergoes a volume change by an external stimulus (provided that, an external stimulus due to contact with a solvent and an external stimulus due to contact with a solution are excluded), the method including:
forming a cover coat layer having a porosity of less than or equal to 40% on a surface of the exposed first nail layer; and
applying the external stimulus to the first nail layer to remove the first nail layer and the cover coat layer from the fingernail.

In another aspect, the present disclosure provides a method of removing a nail adhered to a fingernail, the nail including a first nail layer formed of a resin solidified composition for a nail including a stimulus-responsive material that undergoes a volume change by an external stimulus (provided that, an external stimulus due to contact with a solvent and an external stimulus due to contact with a solution are excluded), and a cover coat layer disposed on the first nail layer and having a porosity of less than or equal to 40%, the method including applying the external stimulus to the first nail layer to remove the first nail layer and the cover coat layer from the fingernail.

### Advantageous Effect of Invention

The cosmetic laminate in the present disclosure can firmly adhere to a part of the living body, and can be easily peeled off from the living body at any timing.

### BRIEF DESCRIPTION OF THE DRAWINGS

FIG. 1 is a schematic cross-sectional view illustrating a state where a cosmetic laminate in a first exemplary embodiment adheres to a living body.

FIG. 2 is a schematic cross-sectional view illustrating a first example of a state where the cosmetic laminate in the first exemplary embodiment is adhered onto a fingernail.

FIG. 3 is a schematic cross-sectional view illustrating a second example of a state where the cosmetic laminate in the first exemplary embodiment is adhered onto a fingernail.

FIG. 4 is a schematic cross-sectional view illustrating a third example of a state where the cosmetic laminate in the first exemplary embodiment is adhered onto a fingernail.

FIG. 5 is a schematic cross-sectional view explaining an example of a principle of a decrease in adhesion strength of the cosmetic laminate to a fingernail when a cosmetic layer of the cosmetic laminate in the first exemplary embodiment is used as a base coat for a gel nail.

FIG. 6 is a schematic cross-sectional view explaining an example of a nail removal method in the first exemplary embodiment.

FIG. 7 is a cross-sectional view illustrating a cover coat layer in a second exemplary embodiment.

FIG. 8 is a schematic cross-sectional view explaining an example of a nail removal method in another exemplary embodiment.

FIG. 9 is a graph showing evaluation results of an adhesion strength ratio before and after irradiation with ultraviolet light of Samples 1 to 3 and Samples 21 to 24.

FIG. 10 is a graph showing a bubble area ratio in the case of Sample 25 without cover coat, with respect to an ultraviolet light transmittance at a wavelength of 340 nm, of Samples 4, 5, 6, 7, 9, 11, and 12.

FIG. 11 is a graph showing a bubble area ratio in the case of Sample 25 without cover coat, with respect to an elastic modulus, of Samples 4, 5, 7, 8, 10, 11, and 12.

### DESCRIPTION OF EMBODIMENTS

Hereinafter, exemplary embodiments will be described in detail with reference to the drawings. However, unnecessary detailed descriptions may be omitted. For example, detailed descriptions of already well-known matters or redundant descriptions of substantially the same configuration may be omitted. This is to avoid an unnecessary redundancy in the following description and to facilitate understanding by those skilled in the art.

Note that, the accompanying drawings and the following description are only presented to help those skilled in the art fully understand the present disclosure, and are not intended to limit the subject matters described in the scope of claims.

### First Exemplary Embodiment

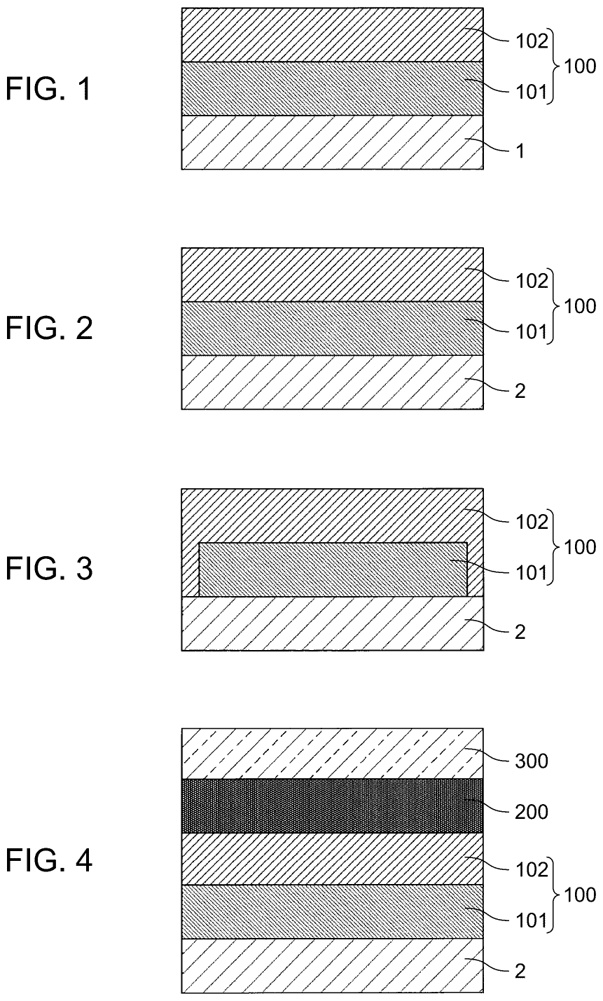

A first exemplary embodiment will be described below with reference to FIGS. 1 to 6.

### [1-1. Configuration]

FIG. 1 is a cross-sectional view illustrating a state where cosmetic laminate 100 in the first exemplary embodiment adheres to living body 1. Cosmetic laminate 100 in the first exemplary embodiment is used as a cosmetic to adhere to a part of living body 1. Cosmetic laminate 100 in first exemplary embodiment includes cosmetic layer 101 adhering to a part of living body 1 and cover coat layer 102. Cosmetic layer 101 and cover coat layer 102 are disposed, cosmetic layer 101 being positioned between living body 1 and cover coat layer 102 in a state where cosmetic laminate 100 is adhered to living body 1. Cosmetic layer 101 contains a resin composition for a cosmetic including a stimulus-responsive material that undergoes a volume change by an external stimulus, and adheres to the part of living body 1 with a solidified matter of the resin composition for a cosmetic. A water contact angle with respect to a surface of the solidified matter of the resin composition for a cosmetic is from 30° to 110°, inclusive. Cosmetic layer 101 reduces an adhesion strength with the part of living body 1 due to the volume change of the solidified matter of the resin composition for a cosmetic caused by application of the external stimulus to the solidified matter of the resin composition for a cosmetic. Cover coat layer 102 has a porosity of less than or equal to 40%. Note that the external stimulus here is an external stimulus excluding an external stimulus due to contact with a solvent and an external stimulus due to contact with a solution. Furthermore, a plurality of stimuli may be combined as the external stimulus.

Since cosmetic laminate 100 in the first exemplary embodiment includes cosmetic layer 101 having the configuration as described above, the cosmetic laminate 100 can firmly adhere to the part of the living body, and can be easily peeled off from the living body at any timing by application of the external stimulus.

In cosmetic laminate 100 in the first exemplary embodiment, cosmetic layer 101 adhering to a part of living body may be a cosmetic that directly adheres to the part of living body, or may be a cosmetic that indirectly adheres to the living body via another layer such as a thin membrane between the cosmetic and the living body. For example, in a case where the cosmetic is a cosmetic for a nail, the thin membrane as the another layer may be, for example, at least one selected from the group consisting of a primer, a base gel, and a base coat.

Cosmetic laminate 100 in the first exemplary embodiment further includes cover coat layer 102. Cover coat layer 102 is disposed, cosmetic layer 101 being positioned between cover coat layer 102 and living body 1. Cover coat layer 102 has a porosity of less than or equal to 40%. In cosmetic laminate 100 in the first exemplary embodiment, in a case where cosmetic layer 101 undergoes a volume change by application of the external stimulus to cosmetic layer 101, a volume change is less likely to occur on a surface of cosmetic layer 101 close to cover coat layer 102, that is, a surface far from living body 1 due to provision of cover coat layer 102. As a result, the force of volume change of cosmetic layer 101 effectively acts on the surface close to living body 1, and cosmetic laminate 100 can be effectively peeled off from living body 1. In other words, since the force of volume change generated in cosmetic layer 101 is sealed by cover coat layer 102 (that is, the force is pressed from above), the force of volume change of cosmetic layer 101 can be efficiently used for interface peeling between living body 1 and cosmetic laminate 100. Thus, cosmetic laminate 100 in the first exemplary embodiment can efficiently reduce the adhesive force with living body 1. Therefore, cosmetic laminate 100 in the first exemplary embodiment can be easily peeled off from the living body by applying the external stimulus at any timing when cosmetic laminate 100 is desired to be peeled off from the living body. Note that the effect of efficiently applying the force of volume change of cosmetic layer 101 to the surface close to living body 1 by cover coat layer 102 as described above may be hereinafter simply referred to as “sealing effect”.

The porosity of cover coat layer 102 is less than or equal to 40%. When the porosity is less than or equal to 40%, an effect of reducing relaxation of the stress acting on the surface close to living body 1 by acting the stress due to the volume change of cosmetic layer 101 on the surface far from living body 1 is obtained. Therefore, cosmetic laminate 100 is easily peeled off from living body 1. In particular, in a case where the volume change of cosmetic layer 101 is volume expansion due to gas, liquid, or the like, cosmetic laminate 100 is more easily peeled off from living body 1 due to the sealing effect by cover coat layer 102. Hereinafter, relaxation of the action of stress due to the volume change of cosmetic layer 101 on the surface close to living body 1 may be simply referred to as “relaxation of stress”.

The porosity of cover coat layer 102 may be less than or equal to 5%. When the porosity of cover coat layer 102 is less than or equal to 5%, the relaxation of stress is further suppressed, and an effect of sealing gas or the like, which causes the volume change in cosmetic layer 101, inside cosmetic layer 101 is also obtained. Therefore, cosmetic laminate 100 is more easily peeled off from living body 1. The porosity of cover coat layer 102 may be less than or equal to 1%. When the porosity of cover coat layer 102 is less than or equal to 1%, the relaxation of stress is further suppressed, and the sealing effect is also improved, so that cosmetic laminate 100 is still more easily peeled off from living body 1.

Pores of the cover coat layer may not be aligned regularly like a mesh and punching. For example, the pores may be pores formed by etching, or may be pores formed in random shapes at random positions formed by process conditions at the time of forming the cover coat layer. In the present disclosure, the pores of the cover coat layer may be pores penetrating the cover coat layer at any position, and may have any shape.

The porosity of cover coat layer 102 can be determined by calculating the area ratio of opening portions in the entire area by area analysis using an image of the main surface of cover coat layer 102 using a photograph or the like of an optical microscope or a microscope.

Cosmetic layer 101 and cover coat layer 102 do not have to be in contact with each other as illustrated in FIG. 1. As an example, in this case, cosmetic layer 101 and cover coat layer 102 are in physical and direct contact with each other. Since cover coat layer 102 is provided in contact with cosmetic layer 101, the sealing effect by cover coat layer 102 can be more effectively exhibited. Therefore, cosmetic laminate 100 is still more easily peeled off from living body 1.

Cosmetic layer 101 and cover coat layer 102 does not have to be in contact with each other. For example, another layer may be provided between cosmetic layer 101 and cover coat layer 102. Also in such a configuration, since the sealing effect by cover coat layer 102 can be obtained, cosmetic laminate 100 is easily peeled off from living body 1.

As long as cover coat layer 102 covers at least a part of cosmetic layer 101, the sealing effect can be obtained. However, in order to more effectively exhibit the sealing effect by cover coat layer 102, cover coat layer 102 desirably covers more than or equal to 50% of the surface area of cosmetic layer 101, more desirably covers more than or equal to 80% of the surface area of cosmetic layer 101, and particularly desirably covers the entire surface of cosmetic layer 101.

Hereinafter, cosmetic layer 101 and cover coat layer 102 will be more specifically described.

### (Cosmetic Layer)

The resin composition for a cosmetic (hereinafter, referred to as “resin composition for a cosmetic in the first exemplary embodiment”) contained in cosmetic layer 101 of cosmetic laminate 100 in the first exemplary embodiment has a water contact angle between 30° and 110° (inclusive) in a solidified state. The contact angle can be measured by using a contact angle meter. The water contact angle is measured by a droplet method. Specifically, an angle between the surface of the solidified matter and the water is obtained by measured with the contact angle meter, for example, in a case where 5 μL of the water is added dropwise to a flat surface of the solidified matter of the resin composition for a cosmetic. The water contact angle can generally represent a permutation of a hydrophilic property and a hydrophobic property of a material, and also has some correlation with physical properties of the material, such as solubility parameters (hereinafter, referred to “SP values”). It is generally known that at least one material selected from the group consisting of a material having similar hydrophilic and hydrophobic properties to each other and a material having the SP values close to each other tend to have good compatibility between the materials as adhesive force increases. A material having a water contact angle between 30° and 110° (inclusive) has a contact angle property close to a contact angle property of the living body such as the fingernail and the lash. Therefore, cosmetic laminate 100 in the first exemplary embodiment has high adhesive force to the living body in a state where the resin composition for a cosmetic is solidified, and can stably adhere to the living body for a long time. In a case where the solidified matter of the resin composition for a cosmetic in the first exemplary embodiment has a water contact angle between 40° and 110° (inclusive), cosmetic laminate 100 in the first exemplary embodiment can more firmly adhere to the living body. In a case where the solidified matter of the resin composition for a cosmetic in the first exemplary embodiment has a water contact angle between 60° and 100° (inclusive), cosmetic laminate 100 in the first exemplary embodiment can more firmly adhere to the living body.

Furthermore, the resin composition for a cosmetic in the first exemplary embodiment includes the stimulus-responsive material undergoing a volume change by the external stimulus, as described above. Since such stimulus-responsive material is included, the solidified matter of the resin composition for a cosmetic in the first exemplary embodiment undergoes a volume change by application of the external stimulus to reduce the adhesion strength with the part of living body. With such configuration, the resin composition for a cosmetic in the first exemplary embodiment can be easily peeled off from the living body by applying the external stimulus at any timing when the composition is desired to be peeled off from the living body. That is, cosmetic layer 101 of cosmetic laminate 100 in the first exemplary embodiment can be easily peeled off from the living body by application of the external stimulus. The stimulus-responsive material may be a material that causes at least one selected from the group consisting of a change in physical property and a chemical reaction by the external stimulus. Examples of the change in physical property include vaporization, sublimation, a change in density, liquefaction, and a change in viscoelasticity. Examples of the chemical reaction include a reaction accompanied with a change in molecular structure and a decomposition reaction. The stimulus-responsive material may be a material that generates a gas due to, for example, the chemical reaction or sublimation caused by the external stimulus. The gas is generated to cause a volume change.

The solidified matter of the resin composition for a cosmetic in the first exemplary embodiment means, for example, a solid such as a coating membrane obtained by volatilizing a solvent of the resin composition for a cosmetic in a case where the resin composition for a cosmetic includes the solvent and is a resin composition capable of forming a solid such as the coating membrane by volatilizing the solvent. Furthermore, in a case where the resin composition for a cosmetic in the first exemplary embodiment is, for example, a curable resin composition that includes a polymerizable compound and can be cured by light or heat, the solidified matter of the resin composition for a cosmetic in the first exemplary embodiment means a cured product of the resin composition for a cosmetic cured by being irradiated with light or heat.

The external stimulus is, for example, at least one selected from the group consisting of light, heat, magnetic force, electricity, and an external force. In accordance with these external stimuli, the peeling can be easier, and direct damage to the living body is less likely to occur. As described above, a plurality of stimuli may be combined as the external stimulus. Therefore, for example, the light and the heat may be simultaneously used as the external stimulus, or a plurality of types of light having wavelengths different from each other may be used. In a case where the light is used as the external stimulus, for example, ultraviolet light, visible light, near infrared light, infrared light, or far infrared light can be used. In a case where the heat is used as the external stimulus, for example, a heat source, hot air, near infrared light, infrared light, far infrared light, or microwave can be used as a way for applying the heat. The external force is, for example, an impact.

A degree of the external stimulus to be applied is not particularly limited. The degree of the external stimulus to be applied to cosmetic laminate 100 may be appropriately adjusted in accordance with at least one selected from the group consisting of a type of the stimulus-responsive material included in the resin composition for a cosmetic in the first exemplary embodiment and a content ratio of the stimulus-responsive material. Furthermore, the degree of the external stimulus to be applied to cosmetic laminate 100 may be changed in accordance with the water contact angle with respect to the surface of the solidified matter of the resin composition for a cosmetic in the first exemplary embodiment.

The external stimulus may be at least one selected from the group consisting of light and heat. The light and the heat can be easily applied with simple equipment. Furthermore, high safety for the living body can be ensured by appropriately selecting a wavelength of the light. For example, a light having a wavelength of more than or equal to 280 nm has the high safety for the living body. Furthermore, the high safety for the living body can be ensured by appropriately selecting a temperature of the heat. A temperature range of the heat capable of being applied as the external stimulus may be appropriately determined in accordance with the part of living body with the cosmetic adhered. For example, in a case where the cosmetic adheres to lashes, for example, the safety for the living body is high at a temperature of less than or equal to 250° C. Furthermore, at a temperature of less than or equal to 100° C., since the safety for not only the lashes but also the living body such as fingernails and skins is higher, many resin compositions for cosmetics to adhere to the living body can be safely utilized as the external stimulus. Therefore, in a case where the external stimulus is at least one selected from the group consisting of light and heat, treatment of peeling off cosmetic laminate 100 from the living body becomes easier, and the direct damage to the living body can be further suppressed.

As the external stimulus, for example, light having a wavelength within a range of less than 400 nm may be used. Here, in the present specification, the phrase “light having a wavelength within a certain specific range” means light including light having a wavelength within the specific range. That is, in a case where the light includes light having a plurality of wavelengths (that is, in the case of light having a spectral distribution), the light may be light including at least light having a wavelength within the specific range, and may further include light having a wavelength outside the specific range. The fact that light having a wavelength within a range of less than 400 nm can be used as the external stimulus can prevent a decrease in adhesion strength of cosmetic laminate 100 to the living body in a normal living environment such as general visible light lighting.

As the external stimulus, for example, light having a wavelength ranging from more than or equal to 280 nm and less than 400 nm may be used. That is, the light used as the external stimulus may include, for example, light having a wavelength ranging from more than or equal to 280 nm and less than 400 nm. The external stimulus may mainly include, for example, a wavelength ranging from more than or equal to 280 nm and less than 400 nm. For example, the wavelength ranging from more than or equal to 280 nm and less than 400 nm may be included in more than or equal to 50% of the total light amount. The light can be easily applied with simple equipment. Furthermore, light having a wavelength of more than or equal to 280 nm has the high safety for the living body. Furthermore, a fact that light having a wavelength of less than 400 nm can be used as the external stimulus can be regarded as that the stimulus-responsive material included in the resin composition for a cosmetic in the first exemplary embodiment is a substance undergoing a volume change with light having the wavelength of less than 400 nm, and is a substance difficultly undergoing a volume change to an extent that the adhesion strength to the living body is reduced with light having a wavelength of more than or equal to 400 nm. Therefore, in this case, it is possible to prevent a decrease in adhesion strength in a normal living environment such as general visible light lighting while implementing application of a stimulus having high energy with a wavelength of about 400 nm. Accordingly, in a case where the light having a wavelength ranging from more than or equal to 280 nm and less than 400 nm is used as the external stimulus, it is possible to implement effective peeling at any timing by a simpler method and to further suppress the damage to the living body while preventing a decrease in adhesion strength of cosmetic laminate 100 to the living body in the normal living environment.

As the external stimulus, light having a peak wavelength ranging from more than or equal to 280 nm and less than 400 nm may be used. In this case, it is possible to implement the effective peeling at any timing by a simpler method and to further suppress the damage to the living body while further preventing a decrease in adhesion strength of cosmetic laminate 100 to the living body in the normal living environment.

In a case where the light having a wavelength ranging from more than or equal to 280 nm and less than 400 nm is used as the external stimulus, the solidified matter of the resin composition for a cosmetic in the first exemplary embodiment may have a transmittance of, for example, more than or equal to 10% for the light with any wavelength ranging from more than or equal to 280 nm and less than 400 nm. Since the solidified matter of the resin composition for a cosmetic in the first exemplary embodiment has such transmittance, the external stimulus effectivity cause the volume change of the stimulus-responsive material, and as a result, the adhesion strength of cosmetic laminate 100 to the part of living body is effectivity reduced. In other words, according to the configuration, cosmetic laminate 100 in the first exemplary embodiment can be effectively peeled off from the living body. The solidified matter of the resin composition for a cosmetic in the first exemplary embodiment may have a transmittance of more than or equal to 30%, or may have a transmittance of more than or equal to 50% for the light with any wavelength ranging from more than or equal to 280 nm and less than 400 nm. In a case where the transmittance is more than or equal to 30%, since the external stimulus cause the volume change of the stimulus-responsive material more effectively, the adhesion strength of cosmetic laminate 100 to the part of living body is reduced more effectively. In a case where the transmittance is more than or equal to 50%, since the external stimulus cause the volume change of the stimulus-responsive material still more effectively, the adhesion strength of cosmetic laminate 100 to the part of living body is reduced still more effectively. As the transmittance is higher, since the external stimulus cause the volume change of the stimulus-responsive material still more effectively, the maximum value of the transmittance is 100%. Furthermore, since the solidified matter of the resin composition for a cosmetic in the first exemplary embodiment has a high transmittance, an effect of increasing transparency and improving designability is also obtained.

The light used as the external stimulus may be, for example, light having a wavelength between 280 nm and 380 nm (inclusive). Since the wavelength of the light applied as the external stimulus is less than or equal to 380 nm, it is possible to more reliably prevent a decrease in adhesion strength in the normal living environment such as the general lighting while implementing application of a stimulus having high energy with a wavelength of about 380 nm. In other words, in a case where the light having a wavelength between 280 nm and 380 nm (inclusive) is used as the external stimulus, it is possible to implement the effective peeling at any timing by a simpler method and to further suppress the damage to the living body while more reliably preventing a decrease in adhesion strength of cosmetic laminate 100 to the living body in the normal living environment. In addition, since light having a wavelength of less than or equal to 380 nm is used as the external stimulus, for example, in a case where the resin composition for a cosmetic in the first exemplary embodiment is used as a base coat agent for a gel nail, an effect that can prevent a decrease in adhesion strength of the base coat during gel nail curing is also obtained. In this case, the solidified matter of the resin composition for a cosmetic in the first exemplary embodiment may, for example, have a transmittance of more than or equal to 10%, or may have a transmittance of more than or equal to 30%, or may have a transmittance of more than or equal to 50% for the light with any wavelength between 280 nm and 380 nm (inclusive). Since the solidified matter of the resin composition for a cosmetic in the first exemplary embodiment has the above transmittance with respect to the wavelength of the light used as the external stimulus, cosmetic laminate 100 in the first exemplary embodiment can be effectively peeled off from the living body by application of the external stimulus.

As the external stimulus, light having a peak wavelength ranging from more than or equal to 280 nm and less than or equal to 380 nm may be used. In this case, it is possible to implement the effective peeling at any timing by a simpler method and to further suppress the damage to the living body while further reliably preventing a decrease in adhesion strength of cosmetic laminate 100 to the living body in the normal living environment.

The light used as the external stimulus may be, for example, light having a wavelength between 280 nm and 370 nm (inclusive), and may be light having a wavelength between 280 nm and 365 nm (inclusive). Furthermore, the light used as the external stimulus may be, for example, light having a peak wavelength between 280 nm and 370 nm (inclusive), and may be light having a peak wavelength between 280 nm and 365 nm (inclusive). When the peak wavelength of the light used as the external stimulus is less than or equal to 370 nm or less than or equal to 365 nm, it is possible to implement the effective peeling at any timing by a simpler method and to further suppress the damage to the living body while further reliably preventing a decrease in adhesion strength of cosmetic laminate 100 to the living body in the normal living environment.

The light used as the external stimulus may be, for example, the light having a peak wavelength between 280 nm and 350 nm (inclusive). Since the peak wavelength of the light used as the external stimulus is less than or equal to 350 nm, irradiation with higher energy causes more volume change. Accordingly, it possible to more effectively peel off cosmetic laminate 100 in the first exemplary embodiment from the living body.

As an example, in a case where the resin composition for a cosmetic in the first exemplary embodiment is cured by light irradiation, light used as the external stimulus (that is, light used for reducing adhesive property of the solidified matter of the resin composition for a cosmetic to the living body) may be, for example, light having a peak wavelength ranging from more than or equal to 280 nm and less than 390 nm. In this case, light used for curing the resin composition for a cosmetic in the present exemplary embodiment by light irradiation may be, for example, between 390 nm and 450 nm (inclusive).

Since the stimulus-responsive material included in the resin composition for a cosmetic in the first exemplary embodiment may be any material that undergoes a volume change by the external stimulus to be applied, the stimulus-responsive material can be appropriately selected in accordance with the external stimulus to be used. However, in consideration of contact with the living body, the stimulus-responsive material is required to have high biocompatibility.

Examples of the stimulus-responsive material undergoing a volume change by the application of light such as at least one selected from the group consisting of ultraviolet light and visible light as the external stimulus include an azide compound such as a glycidyl azide polymer and an azo compound. The azide compound is an organic or inorganic compound having an azide group, and decomposes to generate a nitrogen gas. Furthermore, the azo compound is an organic or inorganic compound having an azo group, and decomposes to generate the nitrogen gas.

Examples of the stimulus-responsive material undergoing the volume change by the application of the heat include
(I) low-boiling materials such as petroleum ether, pentane, hexane, heptane, fatty acid hydrocarbon, methylsilane, and halogen hydrocarbon, and
(II) thermally decomposable foamable materials such as azo compounds, hydrazine derivatives, nitroso compounds, azide compounds, tetrazole compounds, semicarbazide compounds, carbonates, and bicarbonates. For example, the foamable material may be encapsulated with a thermoplastic resin such as (meth)acrylic acid ester, acrylonitrile, and vinylidene chloride in order to prevent volatilization of the stimulus-responsive material at room temperature and to be stably dispersed in the resin composition for a cosmetic. Furthermore, as the stimulus-responsive material undergoing the volume change, a shape memory polymer having a property that the volume is different above and below a glass transition point may be used. Examples of the shape memory polymer include a polyurethane-based shape memory polymer having a volume expanding due to heating at more than or equal to the glass transition point.

The stimulus-responsive material may undergo a volume change by using at least one selected from the group consisting of near infrared light and infrared light as the external stimulus and applying these types of light, and the stimulus-responsive material may undergo a volume change due to heat by using at least one selected from the group consisting of near infrared light and infrared light to increase a temperature of cosmetic laminate 100, specifically, cosmetic layer 101. For example, a material easily absorbing at least one selected from the group consisting of near infrared light and infrared light may be added to the resin composition for a cosmetic. Accordingly, by applying at least one selected from the group consisting of near infrared light and infrared light, an effect of further increasing the temperature of cosmetic laminate 100 may be obtained.

Examples of the material absorbing at least one selected from the group consisting of near infrared light and infrared light to increase the temperature include a cyanine compound, a phthalocyanine compound, a naphthalocyanine-based compound, a porphyrin derivative, a naphthoquinone compound, an anthraquinone compound, a squarylium-based compound, an immonium compound, a diimmonium compound, a triallylmethane-based compound, an azo compound, a dithiol metal complex, and nanoparticles such as carbon or gold.

The content ratio of the stimulus-responsive material in the resin composition for a cosmetic in the first exemplary embodiment can be appropriately selected to enable the volume change that can effectively reduce the adhesion strength of cosmetic laminate 100 to the living body in a case where the external stimulus is applied. For example, the resin composition for a cosmetic in the first exemplary embodiment may include more than or equal to 0.5 vol % of the stimulus-responsive material. Since the stimulus-responsive material is included in an amount of more than or equal to 0.5 vol %, for example, the stimulus-responsive material can be present in an area of more than or equal to 0.5% of an area of an adhesion portion between cosmetic laminate 100, specifically, cosmetic layer 101 and the living body. Accordingly, when the external stimulus is applied, cosmetic layer 101 can undergo a volume change in more than or equal to 0.5% of the area of the adhesion portion between cosmetic layer 101 and the living body. Therefore, when the external stimulus is applied, the adhesion strength of cosmetic laminate 100 to the living body can be effectively reduced, and cosmetic laminate 100 can be more easily peeled off from the living body. The resin composition for a cosmetic in the first exemplary embodiment may include more than or equal to 1 vol % of the stimulus-responsive material. Since the stimulus-responsive material is included in an amount of more than or equal to 1 vol %, when the external stimulus is applied, cosmetic layer 101 can undergo a volume change in more than or equal to 1% of the area of the adhesion portion between cosmetic layer 101 and the living body. Therefore, when the external stimulus is applied, the adhesion strength of cosmetic laminate 100 to the living body can be reduced more effectively, and cosmetic laminate 100 can be still more easily peeled off from the living body.

The resin composition for a cosmetic in the first exemplary embodiment may be composed only of the stimulus-responsive material. In other words, the resin composition for a cosmetic in the first exemplary embodiment may include the stimulus-responsive material at a ratio of 100 vol %. The resin composition for a cosmetic in the first exemplary embodiment may include the stimulus-responsive material at a ratio of less than or equal to 100 vol % or less than or equal to 50 vol %.

The content ratio of the stimulus-responsive material in the resin composition for a cosmetic can be confirmed by, for example, nuclear magnetic resonance (hereinafter, referred to as “NMR”), infrared spectroscopy (hereinafter, referred to as “IR”), mass spectrometry (hereinafter, referred to as “MS”), secondary ion mass spectrometry (hereinafter, referred to as “SIMS”), inductively coupled plasma mass spectrometry (hereinafter, referred to as “ICP-MS”), scanning electron microscopy (hereinafter, referred to as “SEM”), scanning electron microscopy-energy dispersive X-ray spectrometry (hereinafter, referred to as “SEM-EDX”), liquid chromatography, or spectrophotometry. Furthermore, whether or not the stimulus-responsive material is dissolved or uniformly dispersed in the resin composition for a cosmetic can be checked on the basis of transmission uniformity of the light. Whether or not the stimulus-responsive material is dissolved or uniformly dispersed in the resin composition for a cosmetic can also be checked by using a refractive index. In a case where the stimulus-responsive material is a material having a crystal structure, an X-ray diffraction method (hereinafter, referred to as “XRD”) can also be used. In addition, it is also possible to evaluate solubility by using permeability or viscosity of a filter.

The resin composition for a cosmetic in the first exemplary embodiment may include materials other than the stimulus-responsive material. For example, the resin composition for a cosmetic in the first exemplary embodiment may include a polymer material as a base material as another material. The other materials included in the resin composition for a cosmetic in the first exemplary embodiment are selected from, for example, materials having high biocompatibility. For example, the polymer material as the base material may include an acrylic material, a cyanoacrylate-based material, a silicone-based material, a urethane-based material, a urethane(meth)acrylate-based material, a styrene-based material, an epoxy-based material, and an elastomer, having the high biocompatibility. One of these materials may be included, or a plurality of these materials may be mixed and included. In particular, the urethane(meth)acrylate-based material is a main material of a soak off gel nail, and is excellent from a viewpoint of the adhesive property to fingernails, safety, and flexibility. Furthermore, the cyanoacrylate-based material is high in biological safety and adhesive force to lashes or the like. Therefore, in a case where the resin composition for a cosmetic in the first exemplary embodiment is used for a cosmetic to adhere to the lashes, the cyanoacrylate-based material can be suitably used.

Furthermore, the resin composition for a cosmetic in the first exemplary embodiment is a resin composition substantially free of an additive having high toxicity to the living body. Examples of the material having high toxicity to the living body include benzene, ethylene dichloride, acetylene dichloride, monochlorobenzene, ethylbenzene, styrene, lead, mercury, and arsenic. The content of these materials in the resin composition for a cosmetic in the first exemplary embodiment is, for example, less than or equal to 10 ppm, or less than or equal to a detection limit.

The resin composition for a cosmetic in the first exemplary embodiment may have a glass transition point of, for example, less than or equal to 150° C. In a case where the solidified matter undergoes a volume change, the resin composition for a cosmetic being soft to some extent and having the glass transition point of less than or equal to 150° C. obtains an effect of reducing a phenomenon that a large part of the solidified matter is cracked, and easily peeling off cosmetic laminate 100 from the living body.

The resin composition for a cosmetic in the first exemplary embodiment can reduce the adhesion strength of the solidified matter of the resin composition for a cosmetic to the part of living body to, for example, less than or equal to 50% with respect to the adhesion strength before the application of the external stimulus by application of the external stimulus. That is, cosmetic laminate 100 in the first exemplary embodiment can reduce the adhesion strength with the part of living body to, for example, less than or equal to 50% with respect to the adhesion strength before the application of the external stimulus by application of the external stimulus. Since the adhesion strength is reduced to less than or equal to 50%, cosmetic laminate 100 can be more easily peeled off from the living body by applying the external stimulus. The adhesion strength may be reduced to less than or equal to 30% or less than or equal to 10%. As the adhesion strength obtained by applying the external stimulus is lower than the adhesion strength before the application of the external stimulus, that is, initial adhesion strength, both firm adhesion to the living body before the application of the external stimulus and easy peeling after the application of the external stimulus are possible.

The solidified matter of the resin composition for a cosmetic in the first exemplary embodiment undergoes a volume change by application of the external stimulus to reduce the adhesion strength to the part of living body. Such volume change of the solidified matter of the resin composition for a cosmetic occurs, since the stimulus-responsive material undergoes a volume change by application of the external stimulus. A mechanism allowing the stimulus-responsive material to undergo a volume change by application of the external stimulus is not particularly limited. The stimulus-responsive material undergoes a volume change by, for example, causing at least one selected from the group consisting of the change in physical property and the chemical reaction by the external stimulus. Examples of the change in physical property include the vaporization, the sublimation, the change in density, the liquefaction, and the change in viscoelasticity, as described above. Examples of the chemical reaction include the reaction accompanied with the change in molecular structure and the decomposition reaction, as described above. For example, the stimulus-responsive material may undergo a volume change due to a chemical reaction caused by the external stimulus or generation of a gas caused by the sublimation or the like. The volume change of the stimulus-responsive material caused by the external stimulus may be, for example, a volume change of more than or equal to 10%, a volume change of more than or equal to 20%, or a volume change of more than or equal to 50%. Since the volume change of more than or equal to 10% occurs, the adhesion strength easily decreases during the application of the external stimulus. Since the volume change of more than or equal to 20% occurs, the adhesion strength more easily decrease during the application of the external stimulus. Since the volume change of more than or equal to 50% occurs, an effect of more stably reducing the adhesion strength is obtained even with a small stimulus.

For example, the stimulus-responsive material may generate a gas by application of the external stimulus, and therefore, the volume change of the stimulus-responsive material may be caused. Gas generation by the stimulus-responsive material by application of the external stimulus may, for example, occur by a chemical reaction such as decomposition of the stimulus-responsive material by application of the external stimulus, or occur by sublimation of the stimulus-responsive material. Such gas generation causes the volume change of the solidified matter of the resin composition for a cosmetic. Specifically, since bubbles are generated in the solidified matter of the resin composition for a cosmetic by the gas to be generated, the volume change of the solidified matter occurs. Since the volume change of the solidified matter by the generation of the bubbles also occurs at an adhesion interface with the living body, the adhesion area between the solidified material and the living body is reduced, and the adhesion strength decreases.

In a case where the volume change occurs by the gas generation, a volume change rate is large. Therefore, the peeling by application of the external stimulus becomes easier. Examples of the gas to be generated include nitrogen, oxygen, hydrogen, carbon dioxide, water, ammonia, carbon monoxide, nitric acid, and formaldehyde. In particular, in the case of the nitrogen, the carbon dioxide, and the water, the resin composition for a cosmetic is safer since it is excellent in biological safety and is free of flammability.

Furthermore, the application of the external stimulus causes expansion of the stimulus-responsive material, and as a result, the solidified matter of the resin composition for a cosmetic may undergo a volume change. The adhesion strength of the solidified matter to the living body is also reduced due to a volume change caused by expansion of such solidified matter.

In a case where cosmetic layer 101 in the first exemplary embodiment is disposed on the living body, a thickness of cosmetic layer 101 may be, for example, more than or equal to 10 μm. Since the thickness of cosmetic layer 101 is more than or equal to 10 μm, sufficient adhesion strength with the living body is obtained, and in a case where the gas is generated by application of the external stimulus, the gas is less likely to escape in a plane direction, and a larger amount of the gas can be retained between the living body and the solidified matter of the resin composition for a cosmetic.

In a case where the resin composition for a cosmetic in the first exemplary embodiment causes the volume change of the solidified matter of the resin composition for a cosmetic by the gas generation due to the stimulus-responsive material, the solidified matter of the resin composition for a cosmetic may have voids inside. For example, when a plurality of fine voids are present in the solidified matter of the resin composition for a cosmetic, it is considered that the gas easily gathers, since a movement resistance of a void portion is smaller than a movement resistance of a bulk portion (that is, a portion other than the void portion of the resin composition for a cosmetic) in a case where the gas is generated during the application of the external stimulus. Gathering of a large amount of the gas in the void portion causes a larger volume change, and an effect that the solidified matter of the resin composition for a cosmetic is easily peeled off from the living body is obtained. A diameter of the void may be, for example, less than or equal to 50 μm. A plurality of the voids may be present in the solidified matter of the resin composition for a cosmetic. Since many smaller voids (for example, the void having a diameter of less than or equal to 10 μm) are present, it is possible to achieve a large adhesion area with the living body while retaining many voids. Therefore, with such configuration, both stronger adhesion to the living body before the application of the external stimulus and an easier peeling property after the application of the external stimulus can be achieved. Examples of a method for providing the voids in the solidified matter of the resin composition for a cosmetic include a method for including a gas such as air, nitrogen, or carbon dioxide in the resin composition for a cosmetic, and a method for including a porous material having at least one selected from the group consisting of nanopores and micropores in the resin composition for a cosmetic in a state where the gas such as air is included in the pores. Furthermore, when the resin composition for a cosmetic is solidified on the living body, the void can also be provided by generating the gas or the like.

Since the resin composition for a cosmetic in the first exemplary embodiment has a glass transition point of, for example, less than or equal to 150° C., when the gas is generated by the external stimulus, the resin composition for a cosmetic is likely to expand, and the larger volume change occurs. Accordingly, easier peeling becomes possible.

In cosmetic layer 101 in the first exemplary embodiment, in a case where the volume change of the solidified matter of cosmetic layer 101 due to the gas generation from the stimulus-responsive material occurs, the stimulus-responsive material in the resin composition for a cosmetic in the first exemplary embodiment may include the azide compound. The azide compound may be a glycidyl azide polymer. The glycidyl azide polymer has a property of decomposing and releasing the nitrogen gas in a case of irradiation with light in a wavelength region of, for example, about less than or equal to 400 nm. Therefore, in a case where the resin composition for cosmetic in the first exemplary embodiment includes the glycidyl azide polymer, the resin composition for a cosmetic has a high peeling property during the application of the stimulus. Furthermore, the light having a wavelength between 280 nm and 380 nm (inclusive) can be used as the external stimulus. The light in such wavelength range is excellent in safety to the living body. Furthermore, the stimulus-responsive material in response to the light in such wavelength range can prevent the adhesion strength of cosmetic laminate 100 to the living body from being reduced in the normal living environment. In addition, since the light in such wavelength range can be used as the external stimulus, the effective peeling at any timing can be achieved by a simpler method.

In a case where the stimulus-responsive material includes the glycidyl azide polymer, the resin composition for a cosmetic in the first exemplary embodiment may include the glycidyl azide polymer ranging from, for example, 0.5 mass % to 55 mass %, inclusive. Since the glycidyl azide polymer is included in an amount of more than or equal to 0.5 mass %, cosmetic laminate 100 in the first exemplary embodiment can improve the peeling property from the living body during the application of the external stimulus. Since the glycidyl azide polymer is included in an amount of less than or equal to 55 mass %, an effect of easily maintaining physical properties of the polymer material or the like as the base material in the resin composition for a cosmetic is obtained. Therefore, for example, an effect of improving the adhesion strength of cosmetic laminate 100 to the living body before the application of the external stimulus is obtained. A concentration of the glycidyl azide polymer in the resin composition for a cosmetic may be more than or equal to 1 mass %, or more than or equal to 2 mass %. Since the concentration of the glycidyl azide polymer in the resin composition for a cosmetic is more than or equal to 1 mass %, or more than or equal to 2 mass %, cosmetic laminate 100 in the first exemplary embodiment can further improve the peeling property from the living body during the application of the external stimulus. Furthermore, the concentration of the glycidyl azide polymer in the resin composition for a cosmetic may be less than or equal to 35 mass %. Since the concentration of the glycidyl azide polymer in the resin composition for a cosmetic is less than or equal to 35 mass %, cosmetic laminate 100 in the first exemplary embodiment can obtain both properties of a high adhesion before the application of the external stimulus and an excellent peeling property during the application of the external stimulus.

### (Cover Coat Layer)

As described above, cover coat layer 102 is disposed, cosmetic layer 101 being positioned between cover coat layer 102 and living body 1. Cover coat layer 102 has a porosity of less than or equal to 40%.

The tensile modulus of cover coat layer 102 of cosmetic laminate 100 in the first exemplary embodiment is desirably more than or equal to 5 MPa. When the tensile modulus of cover coat layer 102 is more than or equal to 5 MPa, for example, in a case where the volume of cosmetic layer 101 is increased by application of the external stimulus, cosmetic layer 101 is less likely to expand to the surface far from living body 1. As a result, an effect that cosmetic laminate 100 is easily peeled off at the interface with living body 1 is obtained. In particular, in a case where gas is generated by application of the external stimulus, it is considered that an effect that the gas easily spreads in the plane direction of the interface between cosmetic laminate 100 and living body 1 can be obtained. Therefore, an effect that cosmetic laminate 100 is more easily peeled off at the interface with living body 1 is obtained. The tensile modulus of cover coat layer 102 can be determined, for example, by the method in accordance with Japanese Industrial Standard K 7113. Here, under the conditions of the test piece defined in Japanese Industrial Standard K 7113, no pore should be formed in the surface and the peripheral portion of the test piece. However, regarding the tensile modulus of the cover coat layer in the present specification, the tensile modulus is obtained by the method in accordance with Japanese Industrial Standard K 7113 regardless of the porosity of the cover coat layer, that is, even when a pore is provided in the test piece, and the value is shown as the tensile modulus of the cover coat layer. Hereinafter, Japanese Industrial Standard is referred to as “JIS”.

The tensile modulus of cover coat layer 102 of cosmetic laminate 100 in the first exemplary embodiment may be more than or equal to 50 MPa so that cosmetic laminate 100 is more easily peeled off at the interface with living body 1.

Furthermore, the tensile modulus of cover coat layer 102 of cosmetic laminate 100 in the first exemplary embodiment is more desirably more than or equal to 100 MPa. When the tensile modulus is more than or equal to 100 MPa, the force is more easily transmitted in the plane direction, and an easy-peeling effect is obtained.

The tensile modulus of cover coat layer 102 is, for example, less than or equal to 400 GPa.

In cosmetic laminate 100 in the first exemplary embodiment, the adhesion strength between cosmetic layer 101 and cover coat layer 102 is desirably more than or equal to 0.05 N/cm². When the adhesion strength between cosmetic layer 101 and cover coat layer 102 is more than or equal to 0.05 N/cm², even in a case where cosmetic layer 101 undergoes a volume change by application of the external stimulus, adhesion or bonding between cover coat layer 102 and cosmetic layer 101 is easily maintained. As a result, the stress due to the volume change of cosmetic layer 101 can be efficiently applied to the interface between cosmetic laminate 100 and living body 1. Thus, an effect that cosmetic laminate 100 is more easily peeled off at the interface with living body 1 is obtained. As for the adhesion strength in the plane direction between cosmetic layer 101 and cover coat layer 102 in the first exemplary embodiment, cosmetic layer 101 and cover coat layer 102 are bonded in an area of 1 cm², pulled in a direction perpendicular to the bonding surface at 60 mm/min by a tensile tester or the like, and the maximum load at the time of peeling is shown as the adhesion strength. The stronger the adhesive force between cosmetic layer 101 and cover coat layer 102 is, the more desirable it is. In a case where the adhesion strength between cosmetic layer 101 and cover coat layer 102 is, for example, more than or equal to 0.5 N/cm², the stress due to the volume change of cosmetic layer 101 can be more efficiently applied to the interface between cosmetic laminate 100 and living body 1. Thus, an effect that cosmetic laminate 100 is more easily peeled off at the interface with living body 1 is obtained. The adhesion strength between cosmetic layer 101 and cover coat layer 102 is, for example, less than or equal to 100 kN/cm².

In a case where the solidified matter of the resin composition for a cosmetic undergoes a volume change due to generation of a gas by application of the external stimulus, that is, in a case where cosmetic layer 101 undergoes a volume change due to generation of a gas by application of the external stimulus, cover coat layer 102 desirably has a gas permeability of less than or equal to 1.5×10⁸ cc/m²·24 hr·atm. The gas permeability can be measured by, for example, an equal-pressure method (JIS K7126-2), a differential-pressure method (JIS K7126-1), or the like. When the gas permeability of cover coat layer 102 is less than or equal to 1.5×10⁸ cc/m²·24 hr·atm, the gas generated by the external stimulus is less likely to be released to an outside. As a result, an effect that cosmetic laminate 100 is more easily peeled off from living body 1 is obtained. Further, when the gas permeability of the cover coat layer is less than or equal to 1000 cc/m²·24 hr·atm, the gas generated by the external stimulus is further less likely to be released to the outside. As a result, an effect that cosmetic laminate 100 is further easily peeled off from living body 1 is obtained. Cover coat layer 102 does not have to transmit gas at all. Therefore, the gas permeability of cover coat layer 102 is, for example, more than or equal to 0 cc/m²·24 hr·atm.

Cover coat layer 102 in the first exemplary embodiment may contain a polymer material or the like. The material of cover coat layer 102 is selected from, for example, materials having high biocompatibility. For example, the polymer material as the base material of cover coat layer 102 may include an acrylic material, a cyanoacrylate-based material, a silicone-based material, a urethane-based material, a urethane(meth)acrylate-based material, a styrene-based material, an epoxy-based material, and an elastomer, having the high biocompatibility. One of these materials may be contained in cover coat layer 102, or a plurality of these materials may be mixed and contained in cover coat layer 102. In particular, the urethane(meth)acrylate-based material is a main material of a soak off gel nail, and is excellent from a viewpoint of the adhesive property, safety, and the like. Furthermore, cover coat layer 102 may be formed of a film of polyethylene terephthalate (PET), polyethylene, polypropylene, polycarbonate, nylon, cellulose, a cellulose derivative, Teflon (registered trademark), polyimide, acrylic, or the like, or a membrane filter, a nonwoven fabric, a cloth, a mesh, or the like having a porosity of less than or equal to 40%. Furthermore, cover coat layer 102 may be formed of a ceramic material or a metal material. Furthermore, cover coat layer 102 may be a single layer, or may be composed of two or more layers.

In a case where the light having a wavelength ranging from more than or equal to 280 nm and less than 400 nm is used as the external stimulus, cover coat layer 102 may have a transmittance of, for example, more than or equal to 10% for the light with any wavelength ranging from more than or equal to 280 nm and less than 400 nm. Since cover coat layer 102 in the first exemplary embodiment has such transmittance, the external stimulus effectivity cause the volume change of the stimulus-responsive material in the resin composition for a cosmetic, and as a result, the adhesion strength of cosmetic laminate 100 to the part of living body 1 is effectivity reduced. In other words, according to the configuration, cosmetic laminate 100 in the first exemplary embodiment can be effectively peeled off from living body 1. Cover coat layer 102 in the first exemplary embodiment may have a transmittance of more than or equal to 30%, or may have a transmittance of more than or equal to 50% for the light with any wavelength ranging from more than or equal to 280 nm and less than 400 nm. In a case where the transmittance is more than or equal to 30%, since the external stimulus cause the volume change of the stimulus-responsive material more effectively, the adhesion strength of cosmetic laminate 100 to the part of living body 1 is reduced more effectively. In a case where the transmittance is more than or equal to 50%, since the external stimulus cause the volume change of the stimulus-responsive material still more effectively, the adhesion strength of cosmetic laminate 100 to the part of living body 1 is reduced still more effectively. As the transmittance is higher, since the external stimulus cause the volume change of the stimulus-responsive material still more effectively, the maximum value of the transmittance is 100%.

Cover coat layer 102 in the first exemplary embodiment is formed of, for example, a material substantially free of an additive having high toxicity to the living body. Examples of the material having high toxicity to the living body include benzene, ethylene dichloride, acetylene dichloride, monochlorobenzene, ethylbenzene, styrene, lead, mercury, and arsenic. The content of these materials in cover coat layer 102 is, for example, less than or equal to 10 ppm, or less than or equal to a detection limit.

The part of living body that is adhered with cosmetic laminate 100 in the first exemplary embodiment may be at least one selected from the group consisting of the fingernail, the lash, and the skin.

The part of living body that is adhered with cosmetic laminate 100 in the first exemplary embodiment may be at least one selected from the group consisting of the fingernail and the lash. The fingernail or the lash does not have a thin, layered stratum corneum structure that is easily peeled off like the skin. Therefore, by disposing cosmetic laminate 100 in the first exemplary embodiment after a degreasing treatment on the surface is performed, it is possible to obtain a strong adhesion strength capable of causing the nail formed by cosmetic laminate 100 in the first exemplary embodiment or the cosmetic, such as an eyelash extension, which adheres to the living body with cosmetic laminate 100 in the first exemplary embodiment to be continuously disposed on the living body for several weeks. That is, cosmetic laminate 100 in the first exemplary embodiment is suitable for use in a nail or an eyelash extension.

In a case where the part of the living body to which cosmetic laminate 100 in the first exemplary embodiment adheres is a fingernail, the resin composition for a cosmetic contained in cosmetic layer 101 in the first exemplary embodiment may be, for example, a resin composition for a nail that includes a base coat agent for a nail such as a base coat agent for a gel nail, an adhesive for allowing a nail tip to adhere to the own nail, or a nail agent such as a gel nail agent. The resin composition for a nail in the present disclosure may include the resin composition for a cosmetic in the first exemplary embodiment. In this case, the resin composition for a nail of the present disclosure may include a material included in a known resin composition for a nail as a material other than the stimulus-responsive material by appropriate selection according to a use.

In a case where the part of the living body to which cosmetic laminate 100 in the first exemplary embodiment adheres is a fingernail, cosmetic laminate 100 constitutes a nail such as a gel nail to be placed on the fingernail. In this case, for example, cosmetic layer 101 is a first nail layer adhered to the fingernail, and cover coat layer 102 is disposed, the first nail layer being positioned between cover coat layer 102 and the fingernail.

The resin composition for a cosmetic in the first exemplary embodiment is, in particular, suitably used as the base coat agent for a gel nail. That is, in cosmetic laminate 100 in the first exemplary embodiment, for example, the whole may function as a base coat for a gel nail, or cosmetic layer 101 of cosmetic laminate 100 may function as a base coat for a gel nail. As described in the section of BACKGROUND ART, since the gel nail firmly adheres to the own nail by using a photocurable resin or the like, at least one selected from the group consisting of a solvent and polishing is required when the gel nail is peeled off from the fingernail, and the damage to the own nail is very large. However, by using cosmetic laminate 100 in the first exemplary embodiment as the base coat for a gel nail, the gel nail can firmly adhere to the own nail for a necessary period, and by applying the external stimulus when the peeling is desirable, the damage to the own nail can be easily suppressed, and the gel nail and the base coat can be easily peeled off from the own nail.

Hereinafter, an example that the resin composition for a cosmetic according to the first exemplary embodiment is used as the base coat agent for a gel nail will be described.

FIG. 2 is a schematic cross-sectional view illustrating a first example of a state where cosmetic laminate 100 in the first exemplary embodiment is adhered onto fingernail 2. In the example illustrated in FIG. 2, cosmetic laminate 100 is formed by laminating cover coat layer 102 on cosmetic layer 101. In this case, for example, cosmetic layer 101 constitutes a base coat for a gel nail as the first nail layer. Cover coat layer 102 may also serve as a color nail or a top nail.

FIG. 3 is a schematic cross-sectional view illustrating a second example of the state where cosmetic laminate 100 in the first exemplary embodiment is adhered onto fingernail 2. In the example illustrated in FIG. 3, cosmetic laminate 100 is formed by providing cover coat layer 102 to cover the entire surface of cosmetic layer 101. When cover coat layer 102 is placed to cover the entire surface of cosmetic layer 101 in this manner, in a case where a gas is generated by the external stimulus, the gas is less likely to escape from cosmetic layer 101. Therefore, according to this configuration, cosmetic laminate 100 can be more efficiently peeled off from fingernail 2. In this case, for example, cosmetic layer 101 constitutes a base coat for a gel nail as the first nail layer. Cover coat layer 102 may also serve as a color nail or a top nail.

FIG. 4 is a schematic cross-sectional view illustrating a third example of the state where cosmetic laminate 100 in the first exemplary embodiment is adhered onto fingernail 2. In cosmetic laminate 100 of the example illustrated in FIG. 4, cosmetic laminate 100, that is, cosmetic layer 101 and cover coat layer 102, constitutes a base coat for a gel nail. Cosmetic laminate 100 as a base coat is adhered to fingernail 2, and gel nails of color nail 200 and top nail 300 are placed on cosmetic laminate 100. In a case where light such as ultraviolet light is used as the external stimulus, the light transmittance of the color nail is low, and the light may fail to reach fingernail 2 even if the fingernail is irradiated with light. In this case, color nail 200 and top nail 300 are removed by polishing or the like to expose cosmetic laminate 100, and then stimulation such as ultraviolet light can also be applied.

As in the examples illustrated in FIGS. 2 to 4, in a case where cosmetic laminate 100 in the first exemplary embodiment constitutes a nail, such as a gel nail, placed on the fingernail, the nail can be produced by, for example, the production method as described below.

A nail production method in the first exemplary embodiment includes, for example:
applying a resin composition for a nail including a stimulus-responsive material that undergoes a volume change by an external stimulus (provided that, an external stimulus due to contact with a solvent and an external stimulus due to contact with a solution are excluded) to a fingernail, and solidifying the resin composition for a nail to form a first nail layer adhered to the fingernail; and forming a cover coat layer having a porosity of less than or equal to 40% on the first nail layer.

The resin composition for a nail containing the stimulus-responsive material corresponds to the resin composition for a cosmetic in the first exemplary embodiment described in detail above. Furthermore, the details of the material of the cover coat layer and the like are also as described above.

As in the example illustrated in FIG. 4, in a case where a nail in which an additional nail layer such as a color nail has been provided on cosmetic laminate 100 is produced, the production method may further include forming a second nail layer on the cover coat layer. The second nail layer is a color nail or a top nail. A third nail layer may be further formed on the second nail layer. In the example illustrated in FIG. 4, the second nail layer corresponds to color nail 200, and the third nail layer corresponds to top nail 300.

### [1-2. Operation]

In cosmetic laminate 100 in the first exemplary embodiment adhered to the part of the living body, by applying the external stimulus to the solidified matter of the resin composition for a cosmetic contained in cosmetic layer 101, the solidified matter undergoes a volume change. Due to this volume change, the adhesion strength of cosmetic layer 101 to the part of living body 1 is reduced. As described above, cosmetic laminate 100 can be easily peeled off from the living body at any timing due to a decrease in adhesion strength by the application of the external stimulus. Furthermore, since cosmetic laminate 100 in the first exemplary embodiment is provided with cover coat layer 102, the force of volume change of cosmetic layer 101 effectively acts on the surface close to living body 1, and cosmetic laminate 100 can be effectively peeled off from living body 1.

Details such as an example of the stimulus that can be used as the external stimulus are as described above.

Hereinafter, an example of a principle of a decrease in adhesion strength of cosmetic laminate 100 to fingernail 2 when cosmetic layer 101 of cosmetic laminate 100 in the first exemplary embodiment is used as a base coat for a gel nail.

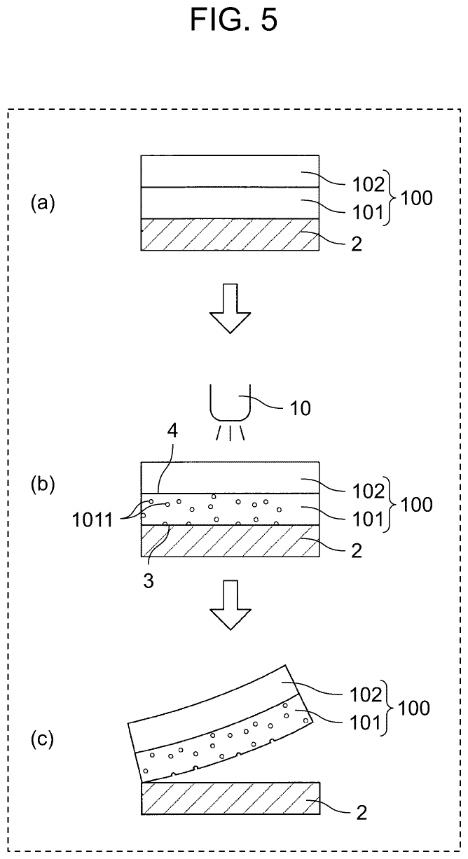

FIG. 5 is a schematic cross-sectional view explaining an example of a principle of a decrease in adhesion strength of cosmetic laminate 100 to fingernail 2 when cosmetic layer 101 of cosmetic laminate 100 in the first exemplary embodiment is used as a base coat for a gel nail as illustrated in FIG. 2. Here, a case where the stimulus-responsive material included in the resin composition for a cosmetic is a material that generates the gas by the external stimulus will be described as an example. As illustrated in part (a) of FIG. 5, before the application of the external stimulus, cosmetic laminate 100 firmly adheres to the surface of fingernail 2 with cosmetic layer 101. As illustrated in part (b) of FIG. 5, when the external stimulus is applied to cosmetic laminate 100 by stimulus generator 10, the stimulus-responsive material included in cosmetic layer 101 generates the gas, bubbles 1011 are generated in cosmetic layer 101, and the volume change of cosmetic layer 101 occurs. Bubbles 1011 are also generated at interface 3 between cosmetic layer 101 and fingernail 2, and also cause the volume change of cosmetic layer 101 at an adhesion portion with fingernail 2, and as a result, a substantial contact area between cosmetic layer 101 and fingernail 2 is reduced. When cosmetic layer 101 undergoes a volume change by application of external stimulus to cosmetic layer 101, a volume change is less likely to occur on a surface of cosmetic layer 101, the surface of cosmetic layer 101 being close to cover coat layer 102, that is, surface 4 far from fingernail 2 due to provision of cover coat layer 102. As a result, the force of volume change of cosmetic layer 101 effectively acts on surface 3 which faces fingernail 2, and cosmetic laminate 100 is effectively peeled off from fingernail 2 as illustrated in part (c) of FIG. 5.

In the case of the example illustrated in FIG. 4, that is, an example having a configuration in which gel nails of color nail 200 and top nail 300 are placed on cosmetic laminate 100, in a case where light such as ultraviolet light is used as the external stimulus, the light transmittance of the color nail is low, and the light may fail to reach sufficiently fingernail 2 even if the fingernail is irradiated. In this case, when cosmetic laminate 100 is peeled off from the living body, parts of color nail 200 and top nail 300 are removed by polishing or the like to expose cosmetic laminate 100, and then stimulation such as ultraviolet light can also be applied. The removal of the color nail and the top nail may be performed on the entire surface, more than or equal to 80% of the area may be removed, more than or equal to 50% of the area may be removed, or more than or equal to 20% of the area may be removed. That is, the nail removal method in such a case is, for example, as follows.

An example of the nail removal method in the first exemplary embodiment is a method of removing a nail which adheres to a fingernail, the nail including a first nail layer formed of a solidified resin composition for a nail including a stimulus-responsive material that undergoes a volume change by an external stimulus (provided that, an external stimulus due to contact with a solvent and an external stimulus due to contact with a solution are excluded), and a cover coat layer disposed on the first nail layer and having a porosity of less than or equal to 40%, the method comprising applying the external stimulus to the first nail layer to remove the first nail layer and the cover coat layer from the fingernail.

The nail further includes at least one other nail layer (for example, layers of color nail 200 and top nail 300) laminated to the first nail layer and the cover coat layer. The method may further include removing at least a part of the at least one other nail layer except the first nail layer and the cover coat layer before applying the external stimulus to the first nail layer.

According to the nail removal method as described above, even in a case where other nail layer such as a color nail is provided on cosmetic laminate 100, cosmetic laminate 100 can be easily peeled off from the fingernail.

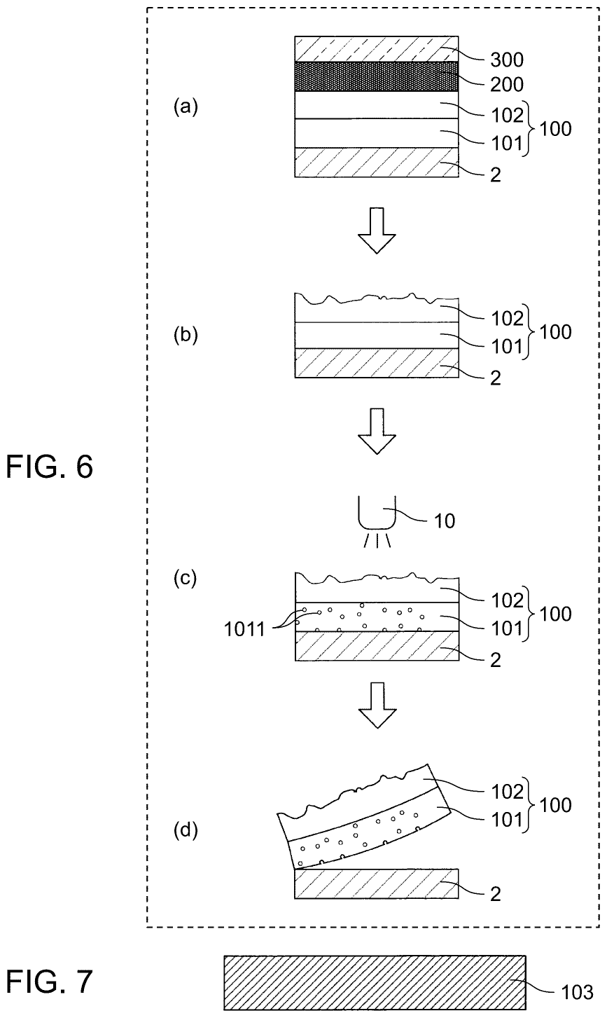

FIG. 6 is a schematic cross-sectional view explaining an example of the nail removal method in the first exemplary embodiment. The example illustrated in FIG. 6 is the example illustrated in FIG. 4, that is, a method of removing a nail in which gel nails of color nail 200 and top nail 300 have been placed on cosmetic laminate 100.

Cosmetic laminate 100 as a base coat adheres to fingernail 2, and gel nails of color nail 200 and top nail 300 are placed on cosmetic laminate 100 (see part (a) of FIG. 6). Cosmetic laminate 100 includes cosmetic layer 101 as the first nail layer and cover coat layer 102. Here, a case where the stimulus-responsive material included in cosmetic layer 101 is a material that generates the gas by the external stimulus will be described as an example. Next, at least parts of color nail 200 and top nail 300 are removed, for example, by polishing, to expose the surface of cosmetic laminate 100, that is, at least a part of cover coat layer 102 (see part (b) of FIG. 6). Next, the external stimulus is applied to cosmetic layer 101 of cosmetic laminate 100 by stimulus generator 10 (see part (c) of FIG. 6). Thus, the stimulus-responsive material included in cosmetic layer 101 generates a gas, bubbles 1011 are generated in cosmetic layer 101, and the volume change of cosmetic layer 101 occurs. As a result, a substantial contact area between cosmetic layer 101 and fingernail 2 is reduced. Thus, the adhesion strength of cosmetic layer 101 to fingernail 2 is reduced, and cosmetic laminate 100 is easily peeled off from fingernail 2 (see part (d) of FIG. 6). The removal of the color nail and the top nail may be performed on the entire surface as in the example illustrated in part (b) of FIG. 6, more than or equal to 80% of the area may be removed, more than or equal to 50% of the area may be removed, or more than or equal to 20% of the area may be removed.

### [1-3. Effects, etc.]

As described above, cosmetic laminate 100 in first exemplary embodiment includes cosmetic layer 101 adhering to a part of living body 1 and cover coat layer 102. Cosmetic layer 101 and cover coat layer 102 are disposed, cosmetic layer 101 being positioned between living body 1 and cover coat layer 102 in a state where cosmetic laminate 100 adheres to living body 1. Cosmetic layer 101 contains a resin composition for a cosmetic including a stimulus-responsive material that undergoes a volume change by an external stimulus (provided that, an external stimulus due to contact with a solvent and an external stimulus due to contact with a solution are excluded), and adheres to the part of living body 1 with a solidified matter of the resin composition for a cosmetic. A water contact angle with respect to a surface of the solidified matter of the resin composition for a cosmetic is from 30° to 110°, inclusive. Cosmetic layer 101 reduces an adhesion strength with the part of living body 1 due to the volume change of the solidified matter of the resin composition for a cosmetic caused by application of the external stimulus to the solidified matter of the resin composition for a cosmetic. Cover coat layer 102 has a porosity of less than or equal to 40%.

According to such configuration, the cosmetic laminate in the first exemplary embodiment can firmly adhere to the part of the living body, and can be easily peeled off from the living body at any timing. Since cosmetic laminate 100 in the first exemplary embodiment includes cover coat layer 102 having a porosity of less than or equal to 40%, a volume change is less likely to occur on a surface of cosmetic layer 101, the surface of cosmetic layer 101 being close to cover coat layer 102, that is, a surface far from living body 1. As a result, the force of volume change of cosmetic layer 101 effectively acts on the surface close to living body 1, and cosmetic laminate 100 can be effectively peeled off from living body 1.

In the first exemplary embodiment, cover coat layer 102 may have a tensile modulus of more than or equal to 5 MPa. When the tensile modulus is more than or equal to 5 MPa, for example, in a case where the volume of cosmetic layer 101 is increased by application of the external stimulus, cosmetic layer 101 is less likely to expand to the surface far from living body 1. As a result, an effect that cosmetic laminate 100 is easily peeled off at the interface with living body 1 is obtained. In particular, in a case where gas is generated by application of the external stimulus, it is considered that an effect that the gas easily spreads in the plane direction of the interface between cosmetic laminate 100 and living body 1 can be obtained. Therefore, an effect that cosmetic laminate 100 is more easily peeled off at the interface with living body 1 is obtained.

In the first exemplary embodiment, the adhesion strength between cosmetic layer 101 and cover coat layer 102 may be more than or equal to 0.05 N/cm². When the adhesion strength between cosmetic layer 101 and cover coat layer 102 is more than or equal to 0.05 N/cm², even in a case where cosmetic layer 101 undergoes a volume change by application of the external stimulus, adhesion or bonding between cover coat layer 102 and cosmetic layer 101 is easily maintained. As a result, the stress due to the volume change of cosmetic layer 101 can be efficiently applied to the interface between cosmetic laminate 100 and living body 1. Thus, an effect that cosmetic laminate 100 is more easily peeled off at the interface with living body 1 is obtained.

In the first exemplary embodiment, in a case where the solidified matter of the resin composition for a cosmetic undergoes a volume change due to generation of a gas by application of the external stimulus, that is, in a case where cosmetic layer 101 undergoes a volume change due to generation of a gas by application of the external stimulus, cover coat layer 102 may have a gas permeability of less than or equal to 1.5×10⁸ cc/m²·24 hr·atm. When the gas permeability of cover coat layer 102 is less than or equal to 1.5×10⁸ cc/m²·24 hr·atm, the gas generated by the external stimulus is less likely to be released to the outside of cosmetic layer 101. As a result, an effect that cosmetic laminate 100 is more easily peeled off from living body 1 is obtained.

In the first exemplary embodiment, the external stimulus may be at least one selected from the group consisting of light, heat, magnetic force, electricity, and external force. According to such configuration, the peeling of cosmetic laminate 100 in the first exemplary embodiment from living body 1 becomes easier, and the direct damage to living body 1 is less likely to occur during the peeling.

In the first exemplary embodiment, the external stimulus may be the light having a wavelength ranging from more than or equal to 280 nm and less than 400 nm. According to such configuration, it is possible to implement the effective peeling at any timing by a simpler method and to further suppress the damage to the living body while preventing a decrease in adhesion strength of cosmetic laminate 100 to living body 1 in the normal living environment.

In the first exemplary embodiment, cover coat layer 102 may have a transmittance of more than or equal to 10% for the light with any wavelength ranging from more than or equal to 280 nm and less than 400 nm. According to such configuration, the external stimulus effectivity cause the volume change of the stimulus-responsive material in the resin composition for a cosmetic, and as a result, the adhesion strength of cosmetic laminate 100 to the part of living body 1 is effectivity reduced. In other words, according to the configuration, cosmetic laminate 100 in the first exemplary embodiment can be effectively peeled off from living body 1.

In the first exemplary embodiment, the stimulus-responsive material may include the azide compound. According to such configuration, cosmetic laminate 100 has the high peeling property during the application of the stimulus, and can also be excellent in safety.

In the first exemplary embodiment, the stimulus-responsive material may include the glycidyl azide polymer. According to such configuration, cosmetic laminate 100 has the high peeling property during the application of the stimulus, and can also be excellent in safety.

In the first exemplary embodiment, cosmetic laminate 100 may include the glycidyl azide polymer ranging from 0.5 mass % to 55 mass %, inclusive. According to such configuration, cosmetic laminate 100 can improve the peeling property from the living body during the application of the external stimulus.

In the first exemplary embodiment, cosmetic layer 101 and cover coat layer 102 may be in contact with each other. Since cover coat layer 102 is provided in contact with cosmetic layer 101, the sealing effect provided by cover coat layer 102 can be more effectively exhibited. Therefore, cosmetic laminate 100 is still more easily peeled off from living body 1.

In the first exemplary embodiment, cosmetic laminate 100 may be used for a nail or an eyelash extension. With such configuration, it is possible to provide a nail or an eyelash extension having a strong adhesion strength capable of causing the nail or the eyelash extension to be continuously disposed on the living body for several weeks.

An example of the nail production method in the first exemplary embodiment includes, for example:
applying a resin composition for a nail including a stimulus-responsive material that undergoes a volume change by an external stimulus (provided that, an external stimulus due to contact with a solvent and an external stimulus due to contact with a solution are excluded) to a fingernail, and solidifying the resin composition for a nail to form a first nail layer adhered to the fingernail; and forming a cover coat layer having a porosity of less than or equal to 40% on the first nail layer. According to this production method, it is possible to provide a nail that can firmly adhere to a fingernail and can be easily peeled off from the fingernail at any timing.

The nail production method may further include, for example, forming a second nail layer on the cover coat layer. The second nail layer is, for example, a color nail or a top nail. According to this method, it is possible to produce a nail in which a further nail layer such as a color nail is provided on cosmetic laminate 100.

The nail removal method in the first exemplary embodiment is, for example, an example of a method of removing a nail adhered to a fingernail, the nail including a first nail layer formed of a solidified resin composition for a nail including a stimulus-responsive material that undergoes a volume change by an external stimulus (provided that, an external stimulus due to contact with a solvent and an external stimulus due to contact with a solution are excluded), and a cover coat layer disposed on the first nail layer and having a porosity of less than or equal to 40%, the method comprising applying the external stimulus to the first nail layer to remove the first nail layer and the cover coat layer from the fingernail. Furthermore, in the above-described nail removal method, the nail may further include at least one other nail layer laminated to the first nail layer and the cover coat layer, and in this case, the method may further include removing the at least one other nail layer except the first nail layer and the cover coat layer before applying the external stimulus to the first nail layer.

According to the nail removal method as described above, even in a case where another nail layer such as a color nail is provided on the cosmetic laminate, the cosmetic laminate can be easily peeled off from the fingernail.

### Second Exemplary Embodiment

A second exemplary embodiment will be described below with reference to FIG. 7.

### [2-1. Configuration]

FIG. 7 is a cross-sectional view illustrating cover coat layer 103 in the second exemplary embodiment. Cover coat layer 103 in the second exemplary embodiment is a cover coat layer covering at least a part of a cosmetic layer that undergoes a volume change by an external stimulus (provided that, an external stimulus due to contact with a solvent and an external stimulus due to contact with a solution are excluded). Cover coat layer 103 has a porosity of less than or equal to 40% and a tensile modulus of more than or equal to 5 MPa.

When cover coat layer 103 having a porosity of less than or equal to 40% in the second exemplary embodiment is placed to cover at least a part of the cosmetic layer that undergoes a volume change by the external stimulus, a volume change can be made less likely to occur on a surface of the cosmetic layer, the surface of the cosmetic layer being close to cover coat layer 103, that is a surface far from the living body. As a result, cover coat layer 103 in the second exemplary embodiment causes the force of volume change of the cosmetic layer to effectively act on the surface close to the living body, and the cosmetic layer can be effectively peeled off from the living body.

In the second exemplary embodiment, cover coat layer 103 has a tensile modulus of more than or equal to 5 MPa. When the tensile modulus is more than or equal to 5 MPa, cover coat layer 103 can suppress expansion of the cosmetic layer to the surface far from the living body, for example, in a case where the volume of the cosmetic layer is increased by application of the external stimulus. As a result, an effect that the cosmetic layer is easily peeled off at the interface with the living body is obtained. In particular, in a case where gas is generated by application of the external stimulus, it is considered that an effect that the gas easily spreads in the plane direction of the interface between cosmetic layer 101 and living body 1 can be obtained. Therefore, an effect that cosmetic laminate 100 is more easily peeled off at the interface with living body 1 is obtained.

In the second exemplary embodiment, cover coat layer 103 may have a transmittance of more than or equal to 10% for the light with any wavelength ranging from more than or equal to 280 nm and less than 400 nm. When cover coat layer 103 in the second exemplary embodiment has such a transmittance, in a case where cover coat layer 103 is placed to cover at least a part of the cosmetic layer that undergoes a volume change by the external stimulus, the volume change of the cosmetic layer can efficiently occur. As a result, cover coat layer 103 in the second exemplary embodiment can efficiently reduce the adhesion strength of the cosmetic layer to the part of the living body. That is, cover coat layer 103 can cause the cosmetic layer to be efficiently peeled off from the living body. Cover coat layer 103 in the second exemplary embodiment may have a transmittance of more than or equal to 30%, or may have a transmittance of more than or equal to 50% for the light with any wavelength ranging from more than or equal to 280 nm and less than 400 nm. In a case where the transmittance is more than or equal to 30%, since the external stimulus cause the volume change of the cosmetic layer more effectively, the adhesion strength of the cosmetic layer to the part of living body is reduced more effectively. In a case where the transmittance is more than or equal to 50%, since the external stimulus cause the volume change of the cosmetic layer still more effectively, the adhesion strength of the cosmetic layer to the part of living body is reduced still more effectively.

Cover coat layer 103 in the second exemplary embodiment corresponds to cover coat layer 102 of cosmetic laminate 100 in the first exemplary embodiment. Therefore, the description of cover coat layer 102 in the first exemplary embodiment can also be applied to cover coat layer 103 in the second exemplary embodiment.

### [2-2. Operation]

Cover coat layer 103 in the second exemplary embodiment can be used for efficiently peeling off, from the living body, the cosmetic layer, which has adhered firmly to the living body and undergoes a volume change by the external stimulus. Cover coat layer 103 in the second exemplary embodiment is placed, for example, to cover at least a part of the cosmetic layer. Cover coat layer 103 in the second exemplary embodiment may be formed on the cosmetic layer immediately after the formation of the cosmetic layer, and may be peeled off from the living body together with the cosmetic layer by applying the external stimulus to the cosmetic layer when the cosmetic layer is desired to be peeled off from the living body after going on the normal life for a while in a state where the cosmetic layer adheres to the living body similarly to cosmetic laminate 100 in the first exemplary embodiment. Alternatively, cover coat layer 103 is placed on the cosmetic layer when the cosmetic layer is peeled off from the living body, and cover coat layer 103 may be used only for peeling off the cosmetic layer from the living body.

### [2-3. Effects, etc.]

As described above, cover coat layer 103 in the second exemplary embodiment is a cover coat layer covering at least a part of a cosmetic layer that undergoes a volume change by the external stimulus. Cover coat layer 103 has a porosity of less than or equal to 40% and a tensile modulus of more than or equal to 5 MPa. With such configuration, cover coat layer 103 in the second exemplary embodiment causes the force of volume change of the cosmetic layer to effectively act on the surface close to the living body, and the cosmetic layer can be effectively peeled off from the living body.

In the second exemplary embodiment, cover coat layer 103 may have a transmittance of more than or equal to 10% for the light with any wavelength ranging from more than or equal to 280 nm and less than 400 nm. With such configuration, in a case where cover coat layer 103 in the second exemplary embodiment is placed to cover at least a part of the cosmetic layer that undergoes a volume change by the external stimulus, the cosmetic layer can be efficiently peeled off from the living body.

### Other Exemplary Embodiments

As described above, the exemplary embodiment has been described as an example of the technique disclosed in the present application. However, the techniques of the present disclosure are not limited thereto, but may be applicable to exemplary embodiments with changes, additions, omissions, or the like. Alternatively, the components described in the above exemplary embodiments may be combined to make an additional exemplary embodiment.

Thus, other exemplary embodiments will be exemplified below.

In the first exemplary embodiment, as the nail removal method, it has been described that cover coat layer 102 is formed on cosmetic layer 101 immediately after cosmetic layer 101 is formed, and after going on the normal life for a while, cover coat layer 102 and cosmetic layer 101 are peeled off from living body 1 together when cosmetic layer 101 is peeled off. However, cover coat layer 102 may be prepared immediately before cosmetic layer 101 is removed.

A method of removing a nail which adheres to a fingernail in a case where cover coat layer 102 is prepared immediately before cosmetic layer 101 is removed is, for example, as follows:
in the nail removal method, the nail includes a first nail layer formed of a solidified resin composition for a nail including a stimulus-responsive material that undergoes a volume change by an external stimulus, and
the method includes:
forming a cover coat layer having a porosity of less than or equal to 40% on a surface of the exposed first nail layer; and
applying the external stimulus to the first nail layer to remove the first nail layer and the cover coat layer from the fingernail.

The nail further includes at least one other nail layer (for example, layers of color nail 200 and top nail 300) laminated to the first nail layer, and
the method may further include removing the at least one other nail layer to expose at least a part of the surface of the first nail layer before forming the cover coat layer.

According to the nail removal method as described above, even in a case where other nail layer such as a color nail is provided on cosmetic laminate 100, cosmetic laminate 100 can be easily peeled off from the fingernail.

As in the nail removal method as described above, by providing the cover coat layer immediately before peeling off the cosmetic layer from the living body, it is possible to provide the cover coat layer specialized in peeling characteristics without being affected by necessary characteristics at the time of use such as long-term adhesion. Therefore, it is easy to achieve both strong adhesion and peeling property.

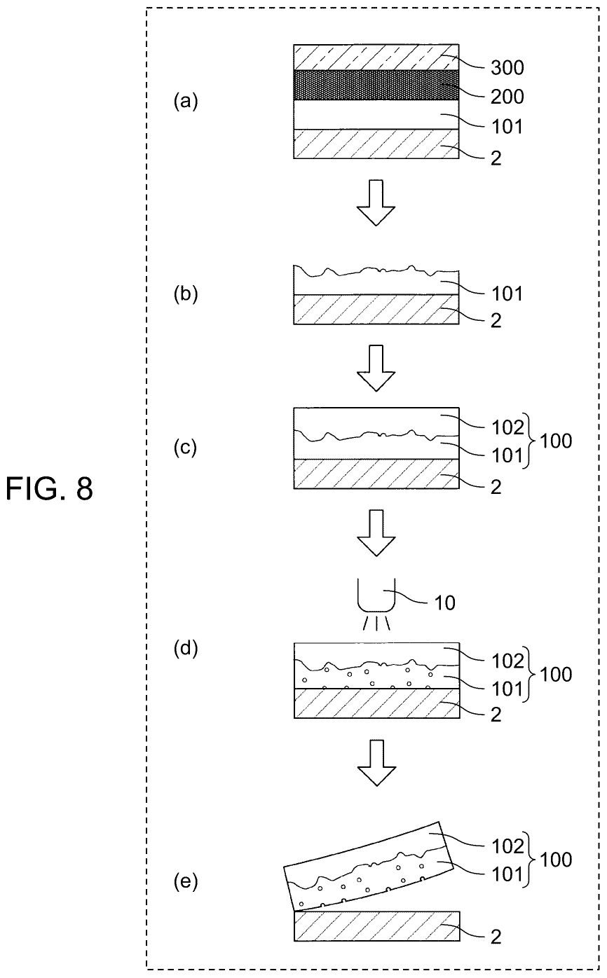

FIG. 8 is a schematic cross-sectional view explaining an example of a nail removal method in another exemplary embodiment.

Cosmetic layer 101 as a base coat adheres to fingernail 2, and gel nails of color nail 200 and top nail 300 are placed on cosmetic layer 101 (see part (a) of FIG. 8). Here, a case where the stimulus-responsive material included in cosmetic layer 101 is a material that generates the gas by the external stimulus will be described as an example. Next, color nail 200 and top nail 300 are removed, for example, by polishing, to expose at least a part of the surface of cosmetic layer 101 (see part (b) of FIG. 8). Next, cover coat layer 102 is formed on cosmetic layer 101 thus exposed (see part (c) of FIG. 8). Next, the external stimulus is applied to cosmetic layer 101 by stimulus generator 10 (see part (d) of FIG. 8). Thus, the stimulus-responsive material included in cosmetic layer 101 generates a gas, bubbles are generated in cosmetic layer 101, and the volume change of cosmetic layer 101 occurs. As a result, a substantial contact area between cosmetic layer 101 and fingernail 2 is reduced. Thus, the adhesion strength of cosmetic layer 101 to fingernail 2 is reduced, and cosmetic layer 101 and cover coat layer 102 are easily peeled off from fingernail 2 (see part (e) of FIG. 8). The removal of the color nail and the top nail may be performed on the entire surface as in the example illustrated in part (b) of FIG. 8, more than or equal to 80% of the area may be removed, more than or equal to 50% of the area may be removed, or more than or equal to 20% of the area may be removed.

In the first exemplary embodiment, cosmetic laminate 100 has been described as an example of the cosmetic laminate of the present disclosure. As described in the first exemplary embodiment, cosmetic laminate 100 in first exemplary embodiment includes cosmetic layer 101 adhering to a part of living body 1 and cover coat layer 102. Cosmetic layer 101 and cover coat layer 102 are disposed, cosmetic layer 101 being positioned between living body 1 and cover coat layer 102 in a state where cosmetic laminate 100 adheres to living body 1. Cosmetic layer 101 contains a resin composition for a cosmetic including a stimulus-responsive material that undergoes a volume change by the external stimulus, and adheres to the part of living body 1 by a solidified matter of the resin composition for a cosmetic. A water contact angle with respect to a surface of the solidified matter of the resin composition for a cosmetic is from 30° to 110°, inclusive. Cosmetic layer 101 reduces an adhesion strength with the part of living body 1 due to the volume change of the solidified matter of the resin composition for a cosmetic caused by application of the external stimulus to the solidified matter of the resin composition for a cosmetic. Cover coat layer 102 has a porosity of less than or equal to 40%.

A cosmetic layer according to another exemplary embodiment of the present disclosure may be a cosmetic laminate having the following configuration in place of cosmetic laminate 100 described above, the cosmetic laminate including:
a cosmetic layer adhering to a part of a living body; and
a cover coat layer,
in which the cosmetic layer and the cover coat layer are disposed, the cosmetic layer being positioned between the living body and the cover coat layer in a state where the cosmetic laminate which adheres to the living body,
the cosmetic layer contains a resin composition for a cosmetic including a stimulus-responsive material that undergoes a volume change due to generation of a gas by an external stimulus (provided that, an external stimulus due to contact with a solvent and an external stimulus due to contact with a solution are excluded), and adheres to the part of the living body with a solidified matter of the resin composition for a cosmetic,
the cosmetic layer reduces an adhesion strength with the part of the living body due to a volume change of the solidified matter caused by the application of the external stimulus to the solidified matter of the resin composition for a cosmetic, and
the cover coat layer has a gas permeability of less than or equal to 1.5×10⁸ cc/m²·24 hr·atm.

In the above-described cosmetic laminate, the water contact angle with respect to the surface of the solidified matter of the resin composition for a cosmetic may be from 30° to 110°, inclusive, so that the adhesive force of the solidified matter of the resin composition for a cosmetic to the living body can be improved, and the solidified matter can stably adhere to the living body for a longer time.

The various configurations of cosmetic laminate 100, cosmetic layer 101, and cover coat layer 102 in the first exemplary embodiment described in the first exemplary embodiment, and the description of the configurations in the first exemplary embodiment can also be applied to the above-described cosmetic laminate.

The exemplary embodiment described above is to exemplify the techniques in the present disclosure, and thus, various modifications, replacements, additions, omissions, and the like can be made in the scope of claims or in an equivalent scope of the claims.

### Note

The above description of the exemplary embodiment discloses the following technologies.

### (Technology 1)

A cosmetic laminate including:
a cosmetic layer adhering to a part of a living body; and
a cover coat layer,
in which the cosmetic layer and the cover coat layer are disposed, the cosmetic layer being positioned between the living body and the cover coat layer in a state where the cosmetic laminate adheres to the living body,
the cosmetic layer contains a resin composition for a cosmetic including a stimulus-responsive material that undergoes a volume change by an external stimulus (provided that, an external stimulus due to contact with a solvent and an external stimulus due to contact with a solution are excluded), and adheres to the part of the living body with a solidified matter of the resin composition for a cosmetic,
a water contact angle with respect to a surface of the solidified matter of the resin composition for a cosmetic is from 30° to 110°, inclusive,
the cosmetic layer reduces an adhesion strength with the part of the living body due to a volume change of the solidified matter caused by application of the external stimulus to the solidified matter of the resin composition for a cosmetic, and
the cover coat layer has a porosity of less than or equal to 40%.

With this configuration, the cosmetic laminate in Technology 1 can firmly adhere to the part of the living body, and can be easily peeled off from the living body at any timing. Since the cosmetic laminate in Technology 1 includes a cover coat layer having a porosity of less than or equal to 40%, a volume change is less likely to occur on a surface of the cosmetic layer, the surface of the cosmetic layer being close to the cover coat layer, that is, a surface far from the living body. As a result, the force of volume change of the cosmetic layer effectively acts on the surface close to the living body, and the cosmetic laminate can be effectively peeled off from the living body.

### (Technology 2)

The cosmetic laminate according to Technology 1, in which the solidified matter of the resin composition for a cosmetic undergoes a volume change due to generation of a gas by application of the external stimulus, and the cover coat layer has a gas permeability of less than or equal to 1.5×10⁸ cc/m²·24 hr·atm.

With this configuration, the cosmetic laminate is more easily peeled off from the living body.

### (Technology 3)

A cosmetic laminate including:
a cosmetic layer adhering to a part of a living body; and
a cover coat layer,
in which the cosmetic layer and the cover coat layer are disposed, the cosmetic layer being positioned between the living body and the cover coat layer in a state where the cosmetic laminate adheres to the living body,
the cosmetic layer contains a resin composition for a cosmetic including a stimulus-responsive material that undergoes a volume change due to generation of a gas by an external stimulus (provided that, an external stimulus due to contact with a solvent and an external stimulus due to contact with a solution are excluded), and adheres to the part of the living body with a solidified matter of the resin composition for a cosmetic,
the cosmetic layer reduces an adhesion strength with the part of the living body due to a volume change of the solidified matter caused by application of the external stimulus to the solidified matter of the resin composition for a cosmetic, and
the cover coat layer has a gas permeability of less than or equal to 1.5×10⁸ cc/m²·24 hr·atm.

With this configuration, the cosmetic laminate in Technology 3 can firmly adhere to the part of the living body, and can be easily peeled off from the living body at any timing. Since the cosmetic laminate in Technology 3 includes a cover coat layer having a gas permeability of less than or equal to 1.5×10⁸ cc/m²·24 hr·atm, the force of volume change of the cosmetic layer effectively acts on the surface close to the living body, and the cosmetic laminate can be effectively peeled off from the living body.

### (Technology 4)

The cosmetic laminate according to Technology 3, in which a water contact angle with respect to a surface of the solidified matter of the resin composition for a cosmetic is from 30° to 110°, inclusive.

With this configuration, the cosmetic laminate in Technology 4 can firmly adhere to the part of living body.

### (Technology 5)

The cosmetic laminate according to any one of Technologies 1 to 4, in which the cover coat layer has a tensile modulus of more than or equal to 5 MPa.

With this configuration, for example, in a case where the volume of the cosmetic layer is increased by application of the external stimulus, the cosmetic layer is less likely to expand to the surface far from the living body. As a result, an effect that the cosmetic laminate is easily peeled off at the interface with the living body is obtained.

### (Technology 6)

The cosmetic laminate according to any one of Technologies 1 to 5, in which an adhesion strength between the cosmetic layer and the cover coat layer is more than or equal to 0.05 N/cm².

With such configuration, the stress due to the volume change of the cosmetic layer can be efficiently applied to the interface between the cosmetic laminate and the living body. Thus, the cosmetic laminate is more easily peeled off at the interface with the living body.

### (Technology 7)

The cosmetic laminate according to any one of Technologies 1 to 6, in which the external stimulus is at least one selected from a group consisting of light, heat, magnetic force, electricity, and external force.

With such configuration, the peeling of the cosmetic laminate from the living body becomes easier, and the direct damage to the living body is less likely to occur during the peeling.

### (Technology 8)

The cosmetic laminate according to Technology 7, in which the external stimulus is light having a wavelength ranging from more than or equal to 280 nm and less than 400 nm.

With such configuration, it is possible to implement the effective peeling at any timing by a simpler method and to further suppress the damage to the living body while preventing a decrease in adhesion strength of the cosmetic laminate to the living body in the normal living environment.

### (Technology 9)

The cosmetic laminate according to Technology 8, in which the cover coat layer has a transmittance of more than or equal to 10% for light having any wavelength ranging from more than or equal to 280 nm and less than 400 nm.

With such configuration, the cosmetic laminate can be efficiently peeled off from the living body.

### (Technology 10)

The cosmetic laminate according to any one of Technologies 1 to 9, in which the stimulus-responsive material includes an azide compound.

With this configuration, the cosmetic laminate has the high peeling property during the application of the stimulus, and can also be excellent in safety.

### (Technology 11)

The cosmetic laminate according to Technology 10, in which the stimulus-responsive material includes a glycidyl azide polymer.

With this configuration, the cosmetic laminate has the high peeling property during the application of the stimulus, and can also be excellent in safety.

### (Technology 12)

The cosmetic laminate according to Technology 11, in which the resin composition for a cosmetic includes the glycidyl azide polymer ranging from 0.5 mass % to 55 mass %, inclusive.

With this configuration, the cosmetic laminate can improve the peeling property from the living body during the application of the external stimulus.

### (Technology 13)

The cosmetic laminate according to any one of Technologies 1 to 12, in which the cosmetic layer and the cover coat layer are in contact with each other.

Since the cover coat layer is provided in contact with the cosmetic layer, the sealing effect provided by the cover coat layer can be more effectively exhibited. Therefore, the cosmetic laminate is still more easily peeled off from the living body.

### (Technology 14)

The cosmetic laminate according to any one of Technologies 1 to 13, in which the cosmetic laminate is used for a nail or an eyelash extension.

With such configuration, it is possible to provide a nail or an eyelash extension having a strong adhesion strength capable of causing the nail or the eyelash extension to be continuously disposed on the living body for several weeks.

### (Technology 15)

A cover coat layer covering at least a part of a cosmetic layer that undergoes a volume change by an external stimulus (provided that, an external stimulus due to contact with a solvent and an external stimulus due to contact with a solution are excluded), the cover coat layer having a porosity of less than or equal to 40% and a tensile modulus of more than or equal to 5 MPa.

With such configuration, the cover coat layer causes the force of volume change of the cosmetic layer to effectively act on the surface close to the living body, and the cosmetic layer can be effectively peeled off from the living body.

### (Technology 16)

The cover coat layer according to Technology 15, in which the cover coat layer has a transmittance of more than or equal to 10% for light having any wavelength ranging from more than or equal to 280 nm and less than 400 nm.

With such configuration, in a case where the cover coat layer is placed to cover at least a part of the cosmetic layer that undergoes a volume change by the external stimulus, the cosmetic layer can be efficiently peeled off from the living body.

### (Technology 17)

A nail production method including:
applying a resin composition for a nail including a stimulus-responsive material that undergoes a volume change by an external stimulus (provided that, an external stimulus due to contact with a solvent and an external stimulus due to contact with a solution are excluded) to a fingernail, and solidifying the resin composition for a nail to form a first nail layer which adheres to the fingernail; and
forming a cover coat layer having a porosity of less than or equal to 40% on the first nail layer.

According to this production method, it is possible to provide a nail that can firmly adhere to a fingernail and can be easily peeled off from the fingernail at any timing.

### (Technology 18)

The method according to Technology 17, further including forming a second nail layer on the cover coat layer, in which the second nail layer is a color nail or a top nail.

According to this method, it is possible to produce a nail in which a further nail layer such as a color nail is provided on a cosmetic laminate.

### (Technology 19)

A method of removing a nail which has adhered to a fingernail, the nail including a first nail layer formed of a solidified resin composition for a nail including a stimulus-responsive material that undergoes a volume change by an external stimulus (provided that, an external stimulus due to contact with a solvent and an external stimulus due to contact with a solution are excluded), the method including:
forming a cover coat layer having a porosity of less than or equal to 40% on an exposed surface of the first nail layer; and
applying the external stimulus to the first nail layer to remove the first nail layer and the cover coat layer from the fingernail.

According to this method, even in a case where other nail layer such as a color nail is provided on the cosmetic laminate, cosmetic laminate 100 can be easily peeled off from the fingernail.

### (Technology 20)

The method according to Technology 19, in which the nail further includes at least one other nail layer laminated to the first nail layer, and
the method further includes removing at least a part of the other nail layer to expose at least a part of the surface of the first nail layer before forming the cover coat layer.

According to this method, even in a case where the other nail layer such as a color nail is provided on the cosmetic laminate, cosmetic laminate 100 can be easily peeled off from the fingernail.

### (Technology 21)

A method of removing a nail which has adhered to a fingernail, the nail including a first nail layer formed of a solidified resin composition for a nail including a stimulus-responsive material that undergoes a volume change by an external stimulus (provided that, an external stimulus due to contact with a solvent and an external stimulus due to contact with a solution are excluded), and a cover coat layer disposed on the first nail layer and having a porosity of less than or equal to 40%, the method including applying the external stimulus to the first nail layer to remove the first nail layer and the cover coat layer from the fingernail.

According to this method, even when another nail layer such as a color nail is provided on the cosmetic laminate, the cosmetic laminate can be easily peeled off from the fingernail.

### (Technology 22)

The method according to Technology 21, in which the nail further includes at least one other nail layer laminated to the first nail layer and the cover coat layer, and the method further includes removing at least a part of the at least one other nail layer except the first nail layer and the cover coat layer before applying the external stimulus to the first nail layer.

According to this method, even when other nail layer such as a color nail is provided on the cosmetic laminate, the cosmetic laminate can be easily peeled off from the fingernail.

### EXAMPLES

The cosmetic laminate of the present disclosure will be described in more detail with reference to examples. The cosmetic laminate of the present disclosure is not limited to the following examples.

[Evaluation of Rate of Change in Adhesion Strength Before and After Irradiation with Ultraviolet Light]

### (Sample 1)

A glycidyl azide polymer (GAP manufactured by NOF CORPORATION) was used as the stimulus-responsive material that undergoes the volume change by application of ultraviolet light. The glycidyl azide polymer has a property of releasing a nitrogen gas by irradiation with light having a wavelength region of about less than or equal to 400 nm.

A resin composition for a cosmetic was prepared by adding a glycidyl azide polymer to a gel nail obtained by mixing 99 mass % of a urethane methacrylate polymer (manufactured by Kyoeisha Chemical Co., Ltd.) and 1 mass % of diphenyl(2,4,6-trimethylbenzoyl) phosphine oxide, the solid content concentration being 20 mass %, and then sufficiently mixing the mixture to be uniform. The fact that the gel nail and the glycidyl azide polymer were sufficiently mixed with each other to be uniform was checked in accordance with the fact that the transmission state of the light was uniform in the resin composition for a cosmetic obtained. Since the density of the glycidyl azide polymer is 1.3 g/cm³ and the density of the commercially available gel nail used is about 1.1 g/cm³, the volume ratio of 20 wt % of the glycidyl azide polymer is 17.5 vol %.

The resin composition for a cosmetic of this sample was applied onto a base material by performing gap coating having a thickness of 100 μm. The obtained coating membrane was irradiated with light emitting diode output light (hereinafter, referred to as “LED output light”) having a wavelength from 390 nm to 400 nm for 2 minutes using a gel nail curing light. Thus, the coating membrane formed of the resin composition for a cosmetic on the base material was cured. As a result, a cosmetic layer formed of the resin composition for a cosmetic was prepared. As the base material, a polyethylene terephthalate film was used. Hereinafter, polyethylene terephthalate is referred to as “PET”. Note that, in general, in a case where a nail is applied to the fingernail, the fingernail is degreased to improve the adhesive property with the fingernail and nail. Since the PET film has a contact angle close to the contact angle of the degreased fingernail, it is considered that the cosmetic laminate of this sample can be evaluated close to the adhesion strength with the fingernail. Here, the water contact angle with respect to the nail surface in a case where the fingernail was degreased was approximately 60° to 75°, whereas a contact angle of the water on the PET film used for evaluation was 62°.

A cover coat layer was prepared by using a gel nail obtained by mixing 99 mass % of a urethane methacrylate polymer (manufactured by Kyoeisha Chemical Co., Ltd.) and 1 mass % of diphenyl(2,4,6-trimethylbenzoyl) phosphine oxide. The gel nail for the cover coat layer was applied onto the cosmetic layer by gap coating having a thickness of 100 μm. The obtained coating membrane was irradiated with LED output light having a wavelength from 390 nm to 400 nm for 2 minutes using a gel nail curing light. Thus, the coating membrane formed of the gel nail on the cosmetic layer was cured. As a result, the cover coat layer formed of the gel nail was prepared, and a cosmetic laminate which adhered to the base material was obtained. The cover coat layer covered the entire surface of the cosmetic layer, and as a result of area analysis by an image using an optical microscope photograph, the porosity was less than 1%. For the subsequent samples, the porosity of the cover coat layer was determined by the same method as in Sample 1.

The ultraviolet light transmittance of the cover coat layer prepared by the above method at a wavelength of 340 nm was 93%, the elastic modulus was 1153 MPa, and the N₂ gas permeability was 2.7 cc/m²·24 hr·atm. The adhesion strength between the cosmetic layer and the cover coat layer was more than or equal to 20 N/cm². Note that the elastic modulus of the cover coat layer was measured by a method in accordance with JIS K 7113. Furthermore, the adhesion strength between the cosmetic layer and the cover coat layer was determined by pulling the cover coat layer at a speed of 60 mm/min in a direction perpendicular to the bonding surface by a tensile tester (MX2-500N-FA manufactured by IMADA Co., Ltd.) and measuring the maximum load at the time of peeling with a digital force gauge (ZTA manufactured by IMADA Co., Ltd.) for a measurement sample in which the cosmetic layer and the cover coat layer were bonded in an area of 1 cm² by the above-described method. The gas permeability of the cover coat layer was measured by a method in accordance with a differential-pressure method (JIS K7126-1). As for the measurement method of the ultraviolet transmittance, measurement was performed using a spectrophotometer (V-770 manufactured by Jasco Corporation). For the subsequent samples, various measurements were performed by the same method as in Sample 1.

The adhesion strength of the cosmetic laminate obtained in this sample was measured. As the measurement sample, a cosmetic laminate which had a size of 10×15 mm and adhered to a base material was used. The adhesion strength in a case where the cosmetic laminate was peeled off from the base material at a speed of 20 mm/min was measured with a digital force gauge (ZTA manufactured by IMADA Co., Ltd.).

In addition, another measurement sample prepared in the same manner was irradiated with the ultraviolet light at 5 J/cm². For irradiation with ultraviolet light, an LED light (manufactured by DOWA) having a center peak at a wavelength of 340 nm was used. An intensity of the ultraviolet light was measured by using a UV LIGHT METER (UV-37SD manufactured by CUSTOM corporation), and an irradiation amount of the ultraviolet light is set to 5 J/cm² by adjusting irradiation time. For the measurement sample after the irradiation with the ultraviolet light at 5 J/cm², the adhesion strength was measured by the same method as in the adhesion strength of the measurement sample without the irradiation with the ultraviolet light. The adhesion strength ratio after the irradiation with the ultraviolet light at 5 J/cm² was 8.4% in a case where the adhesion strength ratio before the irradiation with the ultraviolet light was 100%.

### (Sample 2)

As the cover coat layer, a gel nail obtained by mixing 59 mass % of a urethane methacrylate polymer (manufactured by Kyoeisha Chemical Co., Ltd.), 40 mass % of polyethylene glycol monomethyl ether methacrylate, and 1 mass % of diphenyl(2,4,6-trimethylbenzoyl) phosphine oxide was used. Except for this, a cosmetic laminate of Sample 2 was prepared in the same manner as in Sample 1. The ultraviolet light transmittance of the cover coat layer at a wavelength of 340 nm was 93%, the elastic modulus was 254 MPa, and the N₂ gas permeability was 81 cc/m²·24 hr·atm. The adhesion strength between the cosmetic layer and the cover coat layer was more than or equal to 20 N/cm². The gas permeability of the cover coat layer was measured by a method in accordance with a differential-pressure method (JIS K7126-1).

The cover coat layer covered the entire surface of the cosmetic layer, and the porosity was less than 1%.

The adhesion strength of the cosmetic laminate to the base material before and after the irradiation with the ultraviolet light was evaluated by the same evaluation method as in Sample 1. The adhesion strength ratio after the irradiation with the ultraviolet light of the cosmetic laminate of Sample 2 was 7.4% in a case where the adhesion strength ratio before the irradiation with the ultraviolet light was 100%.

### (Sample 3)

As the cover coat layer, a polycarbonate membrane filter (TSTP14250 manufactured by Merck, porosity: 23%) was used. The polycarbonate membrane filter was placed on the cosmetic layer and then a load of 1 kg weight was applied thereto to bring the membrane filter into close contact with the cosmetic layer. Except for this, a cosmetic laminate of Sample 3 was prepared in the same manner as in Sample 1.

The cover coat layer formed using the polycarbonate membrane filter had an ultraviolet light transmittance of 41% at a wavelength of 340 nm, an elastic modulus of 602 MPa, and a N₂ gas permeability of 1.4×10⁸ cc/m²·24 hr·atm. Furthermore, the adhesion strength between the cosmetic layer and the cover coat layer was 0.6 N/cm². The gas permeability of the cover coat layer was measured by a method in accordance with an equal-pressure method (JIS K7126-2).

The adhesion strength of the cosmetic laminate to the base material before and after the irradiation with the ultraviolet light was evaluated by the same evaluation method as in Sample 1. The adhesion strength ratio after the irradiation with the ultraviolet light was 12% in a case where the adhesion strength ratio before the irradiation with the ultraviolet light was 100%.

### (Sample 21)

A cosmetic layer was prepared using the gel nail used in Sample 1 without the addition of the glycidyl azide polymer. A cover coat layer was not prepared. That is, in Sample 21, only the cosmetic layer was produced using the gel nail used in Sample 1.

The adhesion strength of the cosmetic layer prepared by a gel nail base to the base material before and after the irradiation with the ultraviolet light was evaluated by the same evaluation method as in Sample 1. The adhesion strength ratio after the irradiation with the ultraviolet light was 103% in a case where the adhesion strength ratio before the irradiation with the ultraviolet light was 100%.

### (Sample 22)

A cosmetic layer was prepared on a base material by the same method as in Sample 1. However, a cover coat layer was not formed. That is, in Sample 22, only the cosmetic layer of Sample 1 was formed on the base material.

The adhesion strength of the cosmetic layer to the base material before and after the irradiation with the ultraviolet light was evaluated by the same evaluation method as in Sample 1. The adhesion strength ratio after the irradiation with the ultraviolet light was 30% in a case where the adhesion strength ratio before the irradiation with the ultraviolet light was 100%.

### (Sample 23)

A cosmetic laminate was prepared by the same method as in Sample 3, except that a polyethylene terephthalate mesh (manufactured by AS ONE Corporation, porosity: 63%) was used as the cover coat layer. The adhesion strength between the cosmetic layer and the cover coat layer measured by the same method as in Sample 1 was 0.01 N/cm². The ultraviolet light transmittance of the polyethylene terephthalate mesh at a wavelength of 340 nm was 64%, and the N₂ gas permeability was 2.5×10⁸ cc/m²·24 hr·atm. The elastic modulus of the polyethylene terephthalate mesh was 126 MPa. The gas permeability of the cover coat layer was measured by a method in accordance with an equal-pressure method (JIS K7126-2).

The adhesion strength of the cosmetic laminate to the base material before and after the irradiation with the ultraviolet light was evaluated by the same evaluation method as in Sample 1. The adhesion strength ratio after the irradiation with the ultraviolet light of the cosmetic laminate of Sample 23 was 31% in a case where the adhesion strength ratio before the irradiation with the ultraviolet light was 100%.

### (Sample 24)

A cosmetic laminate was prepared by the same method as in Sample 3, except that a PTFE punching sheet (manufactured by AS ONE Corporation, porosity: 41%) was used as the cover coat layer. The adhesion strength between the cosmetic layer and the cover coat layer measured by the same method as in Sample 1 was 0.03 N/cm². The ultraviolet light transmittance of the PTFE punching sheet at a wavelength of 340 nm was 24%. The elastic modulus of the cover coat layer was 91 MPa.

The adhesion strength of the cosmetic laminate to the base material before and after the irradiation with the ultraviolet light was evaluated by the same evaluation method as in Sample 1. The adhesion strength ratio after the irradiation with the ultraviolet light of the cosmetic laminate of Sample 24 was 36% in a case where the adhesion strength ratio before the irradiation with the ultraviolet light was 100%.

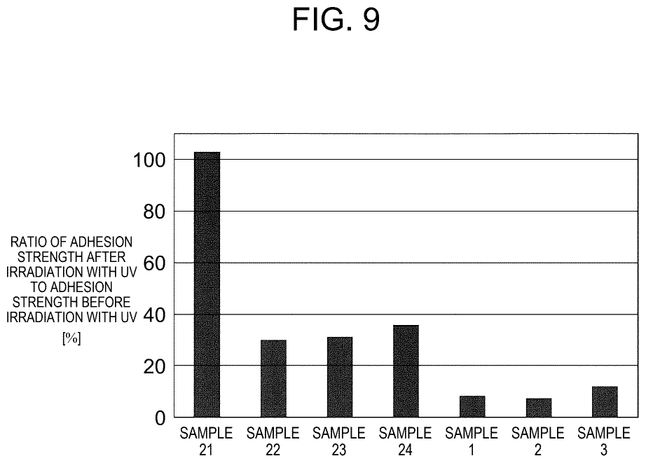

FIG. 9 is a graph showing evaluation results of an adhesion strength ratio before and after irradiation with ultraviolet light of Samples 1 to 3 and Samples 21 to 24. It can be seen that Sample 21 in which the cosmetic layer is prepared by the resin composition for a cosmetic not including a glycidyl azide polymer has almost no change in adhesion strength before and after irradiation with the ultraviolet light. The adhesion strength of Sample 22 was reduced after the irradiation with the ultraviolet light. However, it can be seen that in the cosmetic laminate provided with the cover coat layer having a porosity of less than or equal to 23% in Samples 1 to 3, the adhesion strength after the irradiation with the ultraviolet light is lower than the adhesion strength in Sample 22. In the case of the cover coat layer having a porosity of 63% as in Sample 23 and the case of the cover coat layer having a porosity of 41% as in Sample 24, the effect of further reducing the adhesion strength after irradiation with the ultraviolet light was not obtained with respect to Sample 22 without the cover coat layer.

### [Evaluation of Bubble Area Ratio]

It has been observed by the present inventors that in the cosmetic layer formed by the resin composition for a cosmetic including a glycidyl azide polymer, the peel strength after irradiation with the ultraviolet light tends to decrease as bubbles generated during irradiation with the ultraviolet light are spread in the plane direction of the cosmetic layer. Hereinafter, as compared with a case where the cover coat layer was not provided, how much bubbles spread in the plane direction of the cosmetic layer when the cover coat layer was provided was evaluated.

### (Sample 25)

As the base material, a slide glass was used. A resin composition for a cosmetic was prepared by using PREGEL base gel nail manufactured by PREANFA CO., LTD. containing a urethane acrylate oligomer as a main component, adding a glycidyl azide polymer thereto, the solid content concentration being 20 mass %, and then sufficiently mixing the mixture to be uniform. The fact that the base gel nail and the glycidyl azide polymer were sufficiently mixed with each other to be uniform was checked in accordance with the fact that the transmission state of the light was uniform in the resin composition for a cosmetic obtained. Since the density of the glycidyl azide polymer is 1.3 g/cm³ and the density of the commercially available base gel nail used is about 1.1 g/cm³, the volume ratio of 20 wt % of the glycidyl azide polymer is 17.5 vol %. A cosmetic layer was prepared by the same method as in Sample 1, except that a cosmetic layer having a size of 15 mm×30 mm was prepared on a base material by performing gap coating having a thickness of 200 μm. However, a cover coat layer was not prepared. That is, in Sample 25, only the cosmetic layer was formed on the base material.

The cosmetic layer prepared on the base material was irradiated with ultraviolet light at 10 J/cm². For irradiation with ultraviolet light, an LED light (manufactured by DOWA) having a center peak at a wavelength of 340 nm was used. An intensity of the ultraviolet light was measured by using a UV LIGHT METER (UV-37SD manufactured by CUSTOM corporation), and an irradiation amount of the ultraviolet light is set to 10 J/cm² by adjusting irradiation time. The present inventors observed that bubbles were generated in the cosmetic layer after the irradiation with the ultraviolet light.

### (Sample 4)

As the base material, a slide glass was used. A cosmetic layer having a size of 15 mm×30 mm was prepared on the base material. Thereafter, a cover coat layer was prepared on the cosmetic layer in an area larger than the area of the cosmetic layer. The preparation of the cosmetic layer was the same as in Sample 25, and the preparation method of the cover coat layer was the same as in Sample 1. The adhesion strength between the cosmetic layer and the cover coat layer was more than or equal to 20 N/cm².

The cosmetic laminate composed of the cosmetic layer and the cover coat layer prepared on the base material was irradiated with ultraviolet light at 10 J/cm². For irradiation with ultraviolet light, an LED light (manufactured by DOWA) having a center peak at a wavelength of 340 nm was used. An intensity of the ultraviolet light was measured by using a UV LIGHT METER (UV-37SD manufactured by CUSTOM corporation), and an irradiation amount of the ultraviolet light is set to 10 J/cm² by adjusting irradiation time. The present inventors observed that bubbles were generated in the cosmetic layer after the irradiation with the ultraviolet light. The value obtained by dividing the area of bubbles generated in Sample 4 by the area of bubbles generated in Sample 25, that is, the ratio of the bubble area in the case of Sample 4 with the cover coat layer to the bubble area in the case of the absence of the cover coat layer was 3.8 times. Hereinafter, the ratio of the bubble area in the case of Sample 4 with the cover coat layer to the bubble area in the case of the absence of the cover coat layer is defined as a bubble area ratio. Note that, as for the area of bubbles generated in the cosmetic layer, the area ratio of bubbles was determined by photographing the cosmetic layer and analyzing the image of the bubble generation portion.

### (Sample 5)

As the base material, a slide glass was used. A cosmetic layer having a size of 15 mm×30 mm was prepared on the base material. Thereafter, a cover coat layer was prepared on the cosmetic layer in an area larger than the area of the cosmetic layer. The preparation method of the cosmetic layer was the same as in Sample 25, and the preparation method of the cover coat layer was the same as in Sample 2. The adhesion strength between the cosmetic layer and the cover coat layer was more than or equal to 20 N/cm².

The ratio (bubble area ratio) of the bubble area in the case of Sample 5 with the cover coat layer to the bubble area in the case of Sample 25 without the cover coat layer was determined by the same method as in Sample 4. The bubble area ratio of Sample 5 was 4.1 times.

### (Sample 6)

As the base material, a slide glass was used. A cosmetic layer having a size of 15 mm×30 mm was prepared on the base material. Thereafter, a cover coat layer was prepared on the cosmetic layer in an area larger than the area of the cosmetic layer. The preparation method of the cosmetic layer was the same as in Sample 25, and the preparation method of the cover coat layer was the same as in Sample 3. The adhesion strength between the cosmetic layer and the cover coat layer was 1.0 N/cm².

The ratio (that is, bubble area ratio) of the bubble area in the case of Sample 6 with the cover coat layer to the bubble area in the case of Sample 25 without the cover coat layer was determined by the same method as in Sample 4. The bubble area ratio of Sample 6 was 1.9 times.

### (Sample 7)

A cosmetic laminate was prepared by the same method as in Sample 4, except that a gel nail obtained by mixing 20 mass % of butyl methacrylate and 80 mass % of PRESTO TOPGEL manufactured by Nail Labo was used as the cover coat layer. The ultraviolet light transmittance of the cover coat layer at a wavelength of 340 nm was 87%, the elastic modulus was 329 MPa, and the N₂ gas permeability was 84 cc/m²·24 hr·atm. The adhesion strength between the cosmetic layer and the cover coat layer was more than or equal to 20 N/cm². The gas permeability of the cover coat layer was measured by a method in accordance with a differential-pressure method (JIS K7126-1) and an equal-pressure method (JIS K7126-2).

The cover coat layer covered the entire surface of the cosmetic layer, and the porosity was less than 1%.

The ratio (that is, bubble area ratio) of the bubble area in the case of Sample 7 with the cover coat layer to the bubble area in the case of Sample 25 without the cover coat layer was determined by the same method as in Sample 4. The bubble area ratio of Sample 7 was 3.5 times.

### (Sample 8)

A cosmetic laminate was prepared by the same method as in Sample 4, except that a gel nail obtained by mixing 20 mass % of polyethylene glycol monomethyl ether methacrylate and 80 mass % of PRESTOSOFT TOPGEL manufactured by Nail Labo was used as the cover coat layer. The ultraviolet light transmittance of the cover coat layer at a wavelength of 340 nm was 86%, and the elastic modulus was 6.6 MPa. The adhesion strength between the cosmetic layer and the cover coat layer was more than or equal to 20 N/cm².

The cover coat layer covered the entire surface of the cosmetic layer, and the porosity was less than 1%.

The ratio (that is, bubble area ratio) of the bubble area in the case of Sample 8 with the cover coat layer to the bubble area in the case of Sample 25 without the cover coat layer was determined by the same method as in Sample 4. The bubble area ratio of Sample 8 was 1.8 times.

### (Sample 9)

A cosmetic laminate was prepared by the same method as in Sample 4, except that para gel Clear Gel manufactured by NAIL SELECT was used as the cover coat layer. The ultraviolet light transmittance of the cover coat layer at a wavelength of 340 nm was 35%, and the elastic modulus was 953 MPa. The adhesion strength between the cosmetic layer and the cover coat layer was more than or equal to 20 N/cm².

The cover coat layer covered the entire surface of the cosmetic layer, and the porosity was less than 1%.

The ratio (that is, bubble area ratio) of the bubble area in the case of Sample 9 with the cover coat layer to the bubble area in the case of Sample 25 without the cover coat layer was determined by the same method as in Sample 4. The bubble area ratio of Sample 9 was 1.2 times.

### (Sample 10)

A cosmetic laminate was prepared by the same method as in Sample 4, except that PRESTO SOFT TOPGEL manufactured by Nail Labo was used as the cover coat layer. The ultraviolet light transmittance of the cover coat layer at a wavelength of 340 nm was 82%, and the elastic modulus was 89 MPa. The adhesion strength between the cosmetic layer and the cover coat layer was more than or equal to 20 N/cm². The adhesion strength between the cosmetic layer and the cover coat layer was more than or equal to 20 N/cm².

The cover coat layer covered the entire surface of the cosmetic layer, and the porosity was less than 1%.

The ratio (that is, bubble area ratio) of the bubble area in the case of Sample 10 with the cover coat layer to the bubble area in the case of Sample 25 without the cover coat layer was determined by the same method as in Sample 4. The bubble area ratio of Sample 10 was 1.7 times.

### (Sample 11)

A cosmetic laminate was prepared by the same method as in Sample 6, except that a polyethylene membrane filter (Polyester Track Etched Membrane Disk manufactured by GVS, porosity: 16%) was used as the cover coat layer. The UV transmittance of the cover coat layer at a wavelength of 340 nm was 65%, the elastic modulus was 1703 MPa, and the N₂ gas permeability was 8.2×10⁷ cc/m²·24 hr·atm. Furthermore, the adhesion strength between the cosmetic layer and the cover coat layer was 0.6 N/cm². The gas permeability of the cover coat layer was measured by a method in accordance with an equal-pressure method (JIS K7126-2).

The ratio (that is, bubble area ratio) of the bubble area in the case of Sample 11 with the cover coat layer to the bubble area in the case of Sample 25 without the cover coat layer was determined by the same method as in Sample 4. The bubble area ratio of Sample 11 was 3.1 times.

### (Sample 12)

A cosmetic laminate was prepared by the same method as in Sample 6, except that a polyethylene terephthalate film (Lumirror S10 manufactured by Toray Industries, Inc.) was used as the cover coat layer. The UV transmittance of the cover coat layer at a wavelength of 340 nm was 71%, and the elastic modulus was 2478 MPa. Furthermore, the adhesion strength between the cosmetic layer and the cover coat layer was 0.9 N/cm².

The cover coat layer covered the entire surface of the cosmetic layer, and the porosity was less than 1%.

The ratio (that is, bubble area ratio) of the bubble area in the case of Sample 12 with the cover coat layer to the bubble area in the case of Sample 25 without the cover coat layer was determined by the same method as in Sample 4. The bubble area ratio of Sample 12 was 3.2 times.

### (Sample 13)

A resin composition for a cosmetic was prepared by adding a glycidyl azide polymer to a gel nail obtained by mixing 99 mass % of a urethane methacrylate polymer (manufactured by Kyoeisha Chemical Co., Ltd.) and 1 mass % of diphenyl(2,4,6-trimethylbenzoyl) phosphine oxide, the solid content concentration being 10 mass %, and then sufficiently mixing the mixture to be uniform. A cosmetic laminate was prepared by the same method as in Sample 5, except that the resin composition for a cosmetic was different as described above. Since the density of the glycidyl azide polymer is 1.3 g/cm³ and the density of the gel nail is about 1.1 g/cm³, the volume ratio of 10 wt % of the glycidyl azide polymer is 8.6 mass %.

The adhesion strength between the cosmetic layer and the cover coat layer was more than or equal to 20 N/cm².

The cover coat layer covered the entire surface of the cosmetic layer, and the porosity was less than 1%.

The ratio (that is, bubble area ratio) of the bubble area in the case of Sample 13 with the cover coat layer to the bubble area in the case of Sample 25 without the cover coat layer was determined by the same method as in Sample 4. The bubble area ratio of Sample 13 was 1.6 times.

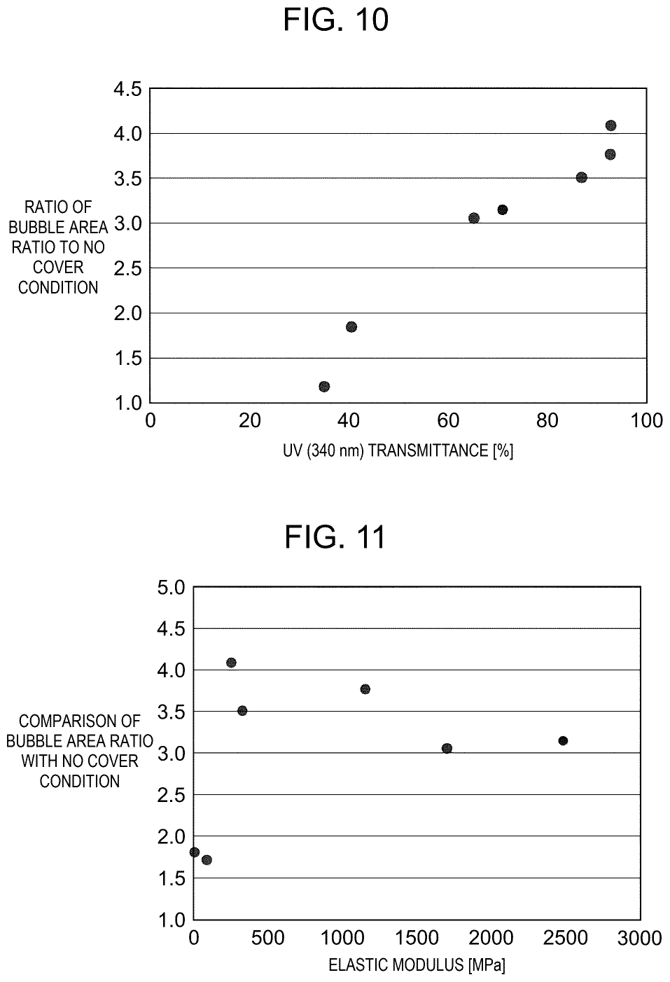

FIG. 10 is a graph showing a bubble area ratio in the case of Sample 25 without cover coat, with respect to an ultraviolet light transmittance at a wavelength of 340 nm, of Samples 4, 5, 6, 7, 9, 11, and 12. Samples 4, 5, 6, 7, 9, 11, and 12 are samples having an elastic modulus of more than 100 MPa among Samples 4 to 10 using the resin composition for a cosmetic in which the solid content concentration of the glycidyl azide polymer is 20 mass %. In the cover coat layer having an elastic modulus of more than or equal to 100 MPa and being hard to some extent, it can be confirmed that the bubbles tend to spread as the ultraviolet light transmittance is higher.

FIG. 11 is a graph showing a bubble area ratio in the case of Sample 25 without cover coat, with respect to an elastic modulus, of Samples 4, 5, 7, 8, 10, 11, and 12. Samples 4, 5, 7, 8, 10, 11, and 12 are samples having an ultraviolet light transmittance at a wavelength of 340 nm of higher than 50% among Samples 4 to 10 using the resin composition for a cosmetic in which the solid content concentration of the glycidyl azide polymer is 20 mass %. When the elastic modulus of the cover coat layer is high (that is, the cover coat layer is hard), it can be confirmed that bubbles tend to spread. As a reason for this, the present inventors believes that in a case where the elastic modulus of the cover coat layer is low (that is, the cover coat layer is soft), when a gas (for example, N₂) is generated by irradiation with ultraviolet light, deformation such as swelling of the cosmetic layer like a balloon in a direction opposite to the living body occurs, and the gas is difficult to spread in the plane direction. On the other hand, in a case where the elastic modulus of the cover coat layer is high (that is, the cover coat layer is hard), it is presumed that the gas efficiently spreads in the plane direction.

### [Evaluation of Adhesive Property to Fingernail]

### (Sample 14)

The resin composition for a cosmetic prepared by the same method as in Sample 7 was applied to the entire surfaces of the fingernails of the human hand with the brush for a nail to form a coating membrane of the resin composition for a cosmetic on the fingernails. This coating membrane was cured by the same method as in Sample 7. As a result, a cosmetic layer formed of the resin composition for a cosmetic was prepared. Further, the cover coat layer used in Sample 7 was placed on the cosmetic layer and cured to attach the cosmetic laminate onto the fingernail. After the curing, the surface was wiped off with ethanol, and then the normal life went on. It was confirmed that the cosmetic laminate was not peeled off from the fingernail for more than or equal to 24 hours.

Furthermore, the resin composition for a cosmetic was applied onto the slide glass by gap coating and cured by the same method as in Sample 7, and then the surface was wiped with ethanol to prepare a film-shaped sheet. The water contact angle with respect to a surface of the film-shaped sheet was evaluated. The contact angle was measured by a droplet method with DROPMASTER DMO-501 manufactured by Kyowa Interface Science Co., Ltd. The contact angle was evaluated by dropping 5 μL of the water. Analysis was performed by a θ/2 method. The contact angle was measured five times to determine an average value. The surface of the film-shaped sheet, that is, the surface of the solidified matter of the resin composition for a cosmetic had a water contact angle of 66.8°.

### (Sample 15)

The resin composition for a cosmetic prepared by the same method as in Sample 13 was applied to the entire surfaces of the fingernails of the human hand with the brush for a nail to form a coating membrane of the resin composition for a cosmetic on the fingernails. This coating membrane was cured by the same method as in Sample 13. As a result, a cosmetic layer formed of the resin composition for a cosmetic was prepared. Further, the cover coat layer used in Sample 13 was placed on the cosmetic layer and cured to attach the cosmetic laminate onto the fingernail. After the curing, the surface was wiped off with ethanol, and then the normal life went on. It was confirmed that the cosmetic laminate was not peeled off from the fingernail for more than or equal to 24 hours. Furthermore, the resin composition for a cosmetic was applied onto the slide glass by gap coating and cured by the same method as in Sample 13, and then the surface was wiped with ethanol to prepare a film-shaped sheet. The water contact angle with respect to a surface of the film-shaped sheet was evaluated by the same method as in Sample 14. The surface of the film-shaped sheet, that is, the surface of the solidified matter of the resin composition for a cosmetic had a water contact angle of 71.6°.

### (Sample 16)

The resin composition for a cosmetic was obtained in the same manner as in Sample 1, except that a commercially available nail “integrate top & base coat” (Shiseido Co., Ltd.) was used, and the glycidyl azide polymer was added, the solid content concentration being 51.7 mass %. The fact that the integrate top & base coat and the glycidyl azide polymer were sufficiently mixed with each other to be uniform was checked in accordance with to the fact that the transmission state of the light was uniform in the resin composition for a cosmetic obtained. Since the main material of the integrate top & base coat is the acrylic-based resin and the density of the acrylic-based resin is generally about 1.2 g/cm³, the volume ratio of the glycidyl azide polymer is 49.7 vol %.

The prepared resin composition for a cosmetic was applied to the entire surfaces of the fingernails of the human hand with the brush for a nail to form a coating membrane of the resin composition for a cosmetic on the fingernails. This coating membrane was naturally dried to prepare a cosmetic layer formed of the resin composition for a cosmetic. Further, the cover coat layer used in Sample 7 was applied and placed onto the cosmetic layer, and the resin composition for a cosmetic on the fingernail was cured by irradiation with LED output light having a wavelength from 390 nm to 400 nm for 2 minutes using a gel nail curing light to attach the cosmetic laminate onto the fingernail. After the curing, the surface was wiped off with ethanol, and then the normal life went on. It was confirmed that the cosmetic laminate was not peeled off from the fingernail for more than or equal to 24 hours.

Furthermore, the resin composition for a cosmetic was applied onto the slide glass by gap coating and naturally dried, and then the surface was wiped with ethanol to prepare a film-shaped sheet. The water contact angle with respect to a surface of the film-shaped sheet was evaluated by the same method as in Sample 14. The surface of the film-shaped sheet, that is, the surface of the solidified matter of the resin composition for a cosmetic had a water contact angle of 100.5°.

### (Sample 17)

A resin composition for a cosmetic was obtained in the same manner as in Sample 1, except that a gel nail prepared by mixing 79.0 mass % of a urethane methacrylate polymer, 20.0 mass % of a quaternary ammonium salt acrylic polymer, and 1.0 mass % of diphenyl(2,4,6-trimethylbenzoyl) phosphine oxide was used, and the glycidyl azide polymer was added, the solid content concentration being 0.65 mass %. The fact that the gel nail and the glycidyl azide polymer were sufficiently mixed with each other to be uniform was checked in accordance with the fact that the transmission state of the light was uniform in the resin composition for a cosmetic obtained. Since the density of the gel nail prepared was 1.0 g/cm³, the volume ratio of the glycidyl azide polymer was 0.5 vol %.

The prepared resin composition for a cosmetic was used to be applied to the entire surfaces of the fingernails of the human hand with the brush for a nail, and the resin composition for a cosmetic was disposed on the fingernails. By using a gel nail curing light to irradiate the resin composition for a cosmetic on the fingernails with LED output light having a wavelength from 390 nm to 400 nm for 2 minutes, the resin composition for a cosmetic on the fingernails was cured. Further, the cover coat layer used in Sample 7 was applied and placed onto the cosmetic layer and cured by the same method as in the resin composition for a cosmetic to attach the cosmetic laminate onto the fingernail. After the curing, the surface was wiped off with ethanol, and then the normal life went on. It was confirmed that the coating membrane cosmetic laminate was not peeled off from the fingernail for more than or equal to 24 hours.

Furthermore, the water contact angle with respect to the surface of the solidified matter of the resin composition for a cosmetic of the present example was measured by the same method as in Sample 14. The water contact angle was 39.6°.

### (Sample 26)

The resin composition for a cosmetic was obtained by the same method as in Sample 1, except that a polyvinyl alcohol adhesive was used, and the glycidyl azide polymer was added and sufficiently mixed, the solid content concentration being 0.54 mass %. The resin composition for a cosmetic of Sample 26 was used to be applied to the entire surfaces of the fingernails of the human hand with the brush for a nail, and the resin composition for a cosmetic was disposed on the fingernails. The resin composition for a cosmetic on the fingernails was sufficiently dried and solidified at room temperature to form the coating membrane as the solidified matter of the resin composition for a cosmetic on the fingernails. Thereafter, as a result of going on the normal life, the coating membrane was peeled off from the fingernail within 1 hour.

Furthermore, the water contact angle with respect to the surface of the solidified matter of the resin composition for a cosmetic of Sample 26 was measured by the same method as in Sample 14. The water contact angle was 26.8°.

### (Sample 27)

The resin composition for a cosmetic was obtained by the same method as in Sample 1, except that a silicone adhesive was used, and the glycidyl azide polymer was added and sufficiently mixed, the solid content concentration being 0.65 mass %. The resin composition for a cosmetic of Sample 27 was used to be applied to the entire surfaces of the fingernails of the human hand with the brush for a nail, and the resin composition for a cosmetic was disposed on the fingernails. The resin composition for a cosmetic on the fingernails was sufficiently dried and solidified at room temperature to form the coating membrane as the solidified matter of the resin composition for a cosmetic on the fingernails. Thereafter, as a result of going on the normal life, the coating membrane was peeled off from the fingernail within 1 hour.

Furthermore, the water contact angle with respect to the surface of the solidified matter of the resin composition for a cosmetic of Sample 27 was measured by the same method as in Sample 14. The water contact angle was 110.6°.

As the evaluation of the adhesive property to the fingernail, after the solidified matter of the resin composition for a cosmetic was disposed on the fingernail, the normal life went on, and a case where the solidified matter was peeled off within 1 hour was evaluated as x, a case where the solidified matter was peeled off within 1 to 24 hours was evaluated as Δ, and a case where the solidified matter was not peeled off for more than or equal to 24 hours was evaluated as ◯. Furthermore, after the resin composition for a cosmetic was disposed on the fingernail, the nail was rubbed 100 times with a cotton swab, and the number of times the solidified matter of the resin composition for a cosmetic started to be peeled off from the fingernail was evaluated. A case where the solidified matter was peeled off within 10 rubs was evaluated as x, a case where the solidified matter was peeled off from 10 to 100 rubs was evaluated as Δ, and a case where the solidified matter was not peeled off even after 100 rubs was evaluated as ◯. The results of Samples 14, 15, 16, 17, 26, and 27 are shown in Table 1 below.

TABLE 1

| | Sample 14 | Sample 15 | Sample 16 | Sample 17 | Sample 26 | Sample 27 |
|---|---|---|---|---|---|---|
| Contact angle | 66.8° | 71.6° | 100.5° | 39.6° | 26.8° | 110.6° |
| 24-Hour peeling | ◯ | ◯ | ◯ | ◯ | X | X |
| property | | | | | | |
| Evaluation of rubbing | ◯ | ◯ | ◯ | ◯ | X | X |
| with cotton swab | (Without | (Without | (Without | (Without | (7 times) | (6 times) |
| (number of times of | peeling) | peeling) | peeling) | peeling) | | |
| peeling) | | | | | | |

As shown in Table 1, the resin compositions for a cosmetic of Samples 14, 15, 16, and 17 in which the water contact angle was in the range between 30° and 110° (inclusive) exhibited higher adhesive property to fingernails than the resin compositions for a cosmetic of Samples 26 and 27 in which the water contact angle was out of the range between 30° and 110° (inclusive).

[Evaluation of Peeling Time from Fingernail]

### (Sample 18)

The resin composition for a cosmetic prepared by the same method as in Sample 7 except that the glycidyl azide polymer was used in an amount of 30 mass % was applied to the entire surfaces of the fingernails of the human hand with the brush for a nail to form a coating membrane of the resin composition for a cosmetic on the fingernails. This coating membrane was cured by the same method as in Sample 7 to form a cosmetic layer. Further, the cover coat layer used in Sample 7 was placed on the cosmetic layer and cured. After the curing, the surface was wiped off with ethanol to prepare a cosmetic laminate. The adhesive force between the cosmetic layer and the cover coat layer was more than or equal to 20 N/cm².

Since the density of the glycidyl azide polymer is 1.3 g/cm³ and the density of the commercially available base gel nail used is about 1.1 g/cm³, the volume ratio of 30 wt % of the glycidyl azide polymer of the resin composition for a cosmetic is 26.6 vol %.

Thereafter, the normal life went on for more than or equal to 6 hours, and it was confirmed that the cosmetic laminate was not peeled off from the fingernail.

Thereafter, the cosmetic laminate on the fingernail was irradiated with ultraviolet light at 15 J/cm². For irradiation with ultraviolet light, an LED light (manufactured by DOWA) having a center peak at a wavelength of 340 nm was used. An intensity of the ultraviolet light was measured by using a UV LIGHT METER (UV-37SD manufactured by CUSTOM corporation), and an irradiation amount of the ultraviolet light is set to 15 J/cm² by adjusting irradiation time. The present inventors observed that bubbles were generated in the cosmetic layer after the irradiation with the ultraviolet light. When the cosmetic laminate was peeled off using a nipper and tweezers for a fingernail, it was confirmed that the cosmetic laminate could be easily peeled off from the fingernail without using an organic solvent such as acetone. The time for peeling off the cosmetic laminate from the fingernail was 20 seconds.

### (Sample 19)

The resin composition for a cosmetic prepared by the same method as in Sample 18 was applied to the entire surfaces of the fingernails of the human hand with the brush for a nail to form a coating membrane of the resin composition for a cosmetic on the fingernails. This coating membrane was cured by the same method as in Sample 18 to form a cosmetic layer. Further, the cover coat layer used in Sample 18 was placed on the cosmetic layer and cured to prepare a cosmetic laminate.

A color gel nail (PREGEL COLOREX S903 manufactured by PREANFA CO., LTD.) was applied to the entire surface of the fingernail on the cover coat layer and cured. Thereafter, a top gel nail (PRESTO TOPGEL manufactured by Nail Labo) was applied to the entire surface of the fingernail on the color gel nail and cured. The curing of the color gel nail and the top gel nail was performed by the same method as in Sample 1, and after the curing of the top gel nail, the surface of the cosmetic laminate was wiped off with ethanol. The normal life went on for more than or equal to 6 hours, and it was confirmed that the cosmetic laminate was not peeled off from the fingernail.

Thereafter, the top gel nail and the color gel nail were removed by polishing. The removal was performed until the color of the color layer disappeared and became transparent in an area of more than or equal to 90%. After this removal step, the cosmetic laminate whose surface is slightly polished is placed on the fingernail.

the cosmetic laminate (that is, the cosmetic laminate having a slightly polished surface) on the fingernail was irradiated with ultraviolet light at 15 J/cm². For irradiation with ultraviolet light, an LED light (manufactured by DOWA) having a center peak at a wavelength of 340 nm was used. An intensity of the ultraviolet light was measured by using a UV LIGHT METER (UV-37SD manufactured by CUSTOM corporation), and an irradiation amount of the ultraviolet light is set to 15 J/cm² by adjusting irradiation time. The present inventors observed that bubbles were generated in the cosmetic layer after the irradiation with the ultraviolet light. When the cosmetic laminate was peeled off using a nipper and tweezers for a fingernail, it was confirmed that the cosmetic laminate could be easily peeled off from the fingernail without using an organic solvent such as acetone. The time for peeling off the cosmetic laminate from the fingernail was 14 seconds.

### (Sample 20)

The resin composition for a cosmetic prepared by the same method as in Sample 18 was applied to the entire surfaces of the fingernails of the human hand with the brush for a nail to form a coating membrane of the resin composition for a cosmetic on the fingernails. This coating membrane was cured by the same method as in Sample 18 to form a cosmetic layer. Thereafter, a color gel nail (PREGEL COLOREX S903 manufactured by PREANFA CO., LTD.) was applied to the entire surface of the fingernail on the cosmetic layer and cured, and then a top coat (TOPGEL manufactured by Nail Labo) was applied to the entire surface of the fingernail and the cured. The curing of the color gel nail and the top gel nail was performed by the same method as in Sample 1, and after the curing of the top gel nail, the surface was wiped off with ethanol. The normal life went on for more than or equal to 6 hours, and it was confirmed that the laminate of the cosmetic layer, the color gel nail, and the top gel nail was not peeled off from the fingernail.

Thereafter, the top gel nail and the color gel nail were removed by polishing. The removal was performed until the color of the color layer disappeared and became transparent in an area of more than or equal to 90%. After this removal step, the cosmetic layer is substantially exposed on the fingernail. The exposed surface of the cosmetic layer was in a state of being slightly polished. The sample cover coat layer as in Sample 7 was prepared on such a cosmetic layer by the same method as in Sample 7. The adhesion strength between the cosmetic layer and the cover coat layer was more than or equal to 20 N/cm².

The cover coat layer covered the entire surface of the cosmetic layer, and the porosity was less than 1%.

At this time, the cosmetic laminate including the cosmetic layer and the cover coat layer is formed on the fingernail.

The cosmetic laminate on the fingernail was irradiated with ultraviolet light at 15 J/cm². For irradiation with ultraviolet light, an LED light (manufactured by DOWA) having a center peak at a wavelength of 340 nm was used. An intensity of the ultraviolet light was measured by using a UV LIGHT METER (UV-37SD manufactured by CUSTOM corporation), and an irradiation amount of the ultraviolet light is set to 15 J/cm² by adjusting irradiation time. The present inventors observed that bubbles were generated in the cosmetic layer after the irradiation with the ultraviolet light. When the cosmetic laminate was peeled off using a nipper and tweezers for a fingernail, it was confirmed that the cosmetic laminate could be easily peeled off from the fingernail without using an organic solvent such as acetone. The time for peeling off the cosmetic laminate from the fingernail was 25 seconds.

### (Sample 28)

The resin composition for a cosmetic prepared by the same method as in Sample 18 was applied to the entire surfaces of the fingernails of the human hand with the brush for a nail to form a coating membrane of the resin composition for a cosmetic on the fingernails. This coating membrane was cured by the same method as in Sample 18 to form a cosmetic layer. The surface of the cosmetic layer was wiped off with ethanol. A cover coat layer was not placed.

The cosmetic layer on the fingernail was irradiated with ultraviolet light at 15 J/cm². For irradiation with ultraviolet light, an LED light (manufactured by DOWA) having a center peak at a wavelength of 340 nm was used. An intensity of the ultraviolet light was measured by using a UV LIGHT METER (UV-37SD manufactured by CUSTOM corporation), and an irradiation amount of the ultraviolet light is set to 15 J/cm² by adjusting irradiation time. The present inventors observed that bubbles were generated to some extent in the cosmetic layer after the irradiation with the ultraviolet light. When the cosmetic layer was peeled off using a nipper and tweezers for a fingernail, it was confirmed that the cosmetic layer could be peeled off from the fingernail without using an organic solvent such as acetone, but the time for peeling off the cosmetic layer from the fingernail was required to be more than or equal to 300 seconds.

### (Sample 29)

The gel nail base used in Sample 21 was applied to the entire surfaces of the fingernails of the human hand with the brush for a nail, and the coating membrane was cured in the same manner as in Sample 21 to prepare a cosmetic layer composed of the gel nail base on the fingernails. The surface of the cosmetic layer was wiped off with ethanol.

Thereafter, the cosmetic layer on the fingernail was irradiated with ultraviolet light by the same method as in Sample 18. No bubbles were observed in the cosmetic layer after the irradiation with the ultraviolet light, and the gel nail as the cosmetic layer could not be peeled off from the fingernail without swelling by an organic material such as acetone.

Table 2 shows the results of the peeling property of the cosmetic laminate or the cosmetic layer after the irradiation with the ultraviolet light of Samples 18, 19, 20, 28, and 29. A case where the cosmetic laminate or the cosmetic layer can be peeled off within 300 seconds without an organic solvent such as acetone after the irradiation with the ultraviolet light is indicated by ◯, a case where the cosmetic laminate or the cosmetic layer can be peeled off without an organic solvent such as acetone although it takes a time of more than or equal to 300 seconds is indicated by Δ, and a case where the cosmetic laminate or the cosmetic layer cannot be peeled off without an organic solvent such as acetone is indicated by x.

From Table 2, in Samples 18, 19, and 20 with the cover coat layer, the cosmetic laminate could be easily peeled off from the fingernail in an early time within 30 seconds without an organic solvent such as acetone. On the other hand, for Sample 28 including the cosmetic layer formed by the resin composition for a cosmetic including a stimulus-responsive material but not provided with the cover coat layer, the cosmetic layer could be peeled off without an organic solvent, but it took more than or equal to 300 seconds. Furthermore, for Sample 29 in which the cosmetic layer was formed by a composition not including a stimulus-responsive material and the cover coat layer was also not provided, peeling without an organic solvent was not possible.

TABLE 2

| | Sample 18 | Sample 19 | Sample 20 | Sample 28 | Sample 29 |
|---|---|---|---|---|---|
| Peeling property after | ∘ | ∘ | ∘ | Δ | x |
| irradiation with ultraviolet light | | | | | |
| Time necessary for peeling off | 20 seconds | 14 seconds | 25 seconds | More than | Not |
| cosmetic layer or cosmetic | | | | or equal to | peelable |
| laminate from fingernail | | | | 300 seconds | |
| without acetone | | | | | |

### INDUSTRIAL APPLICABILITY

The resin composition for a cosmetic of the present disclosure can be used for, for example, the base coat of the nail. Furthermore, the resin composition for a cosmetic of the present disclosure can also be used for use of, for example, the adhesive for an eyelash extension and the adhesive for a lash.

### REFERENCE MARKS IN THE DRAWINGS

1 living body
2 fingernail
100 cosmetic laminate
101 cosmetic layer
102, 103 cover coat layer
200 color nail
300 top nail

1. A cosmetic laminate comprising:
a cosmetic layer adhering to a part of a living body; and
a cover coat layer,
wherein
the cosmetic layer and the cover coat layer are disposed, the cosmetic layer being positioned between the living body and the cover coat layer in a state where the cosmetic laminate adheres to the living body,
the cosmetic layer contains a resin composition for a cosmetic including a stimulus-responsive material that undergoes a volume change by an external stimulus (provided that, an external stimulus due to contact with a solvent and an external stimulus due to contact with a solution are excluded), and adheres to the part of the living body with a solidified matter of the resin composition for a cosmetic,
a water contact angle with respect to a surface of the solidified matter of the resin composition for a cosmetic is from 30° to 110°, inclusive,
the cosmetic layer reduces an adhesion strength with the part of the living body by a volume change of the solidified matter caused by application of the external stimulus to the solidified matter of the resin composition for a cosmetic, and
the cover coat layer has a porosity of less than or equal to 40%.

2. The cosmetic laminate according to claim 1, wherein
the solidified matter of the resin composition for a cosmetic undergoes a volume change due to generation of a gas by application of the external stimulus, and
the cover coat layer has a gas permeability of less than or equal to 1.5×10⁸ cc/m²·24 hr·atm.

3. A cosmetic laminate comprising:
a cosmetic layer adhering to a part of a living body; and
a cover coat layer,
wherein the cosmetic layer and the cover coat layer are disposed, the cosmetic layer being positioned between the living body and the cover coat layer in a state where the cosmetic laminate adheres to the living body,
the cosmetic layer contains a resin composition for a cosmetic including a stimulus-responsive material that undergoes a volume change due to generation of a gas by an external stimulus (provided that, an external stimulus due to contact with a solvent and an external stimulus due to contact with a solution are excluded), and adheres to the part of the living body with a solidified matter of the resin composition for a cosmetic,
the cosmetic layer reduces an adhesion strength with the part of the living body by a volume change of the solidified matter caused by application of the external stimulus to the solidified matter of the resin composition for a cosmetic, and
the cover coat layer has a gas permeability of less than or equal to 1.5×10⁸ cc/m²·24 hr·atm.

4. The cosmetic laminate according to claim 3, wherein
a water contact angle with respect to a surface of the solidified matter of the resin composition for a cosmetic is from 30° to 110°, inclusive.

5. The cosmetic laminate according to claim 1, wherein
the cover coat layer has a tensile modulus of more than or equal to 5 MPa.

6. The cosmetic laminate according to claim 1, wherein
an adhesion strength between the cosmetic layer and the cover coat layer is more than or equal to 0.05 N/cm².

7. The cosmetic laminate according to claim 1, wherein
the external stimulus is at least one selected from a group consisting of light, heat, magnetic force, electricity, and external force.

8. The cosmetic laminate according to claim 7, wherein
the external stimulus is light having a wavelength ranging from more than or equal to 280 nm and less than 400 nm.

9. The cosmetic laminate according to claim 8, wherein
the cover coat layer has a transmittance of more than or equal to 10% for light having any wavelength ranging from more than or equal to 280 nm and less than 400 nm.

10. The cosmetic laminate according to claim 1, wherein
the stimulus-responsive material includes an azide compound.

11. The cosmetic laminate according to claim 10, wherein
the stimulus-responsive material includes a glycidyl azide polymer.

12. The cosmetic laminate according to claim 11, wherein
the resin composition for a cosmetic includes the glycidyl azide polymer ranging from 0.5 mass % to 55 mass %, inclusive.

13. The cosmetic laminate according to claim 1- or 3, wherein
the cosmetic layer and the cover coat layer are in contact with each other.

14. The cosmetic laminate according to claim 1- or 3, wherein
the cosmetic laminate is used for a nail or an eyelash extension.

15. A cover coat layer covering at least a part of a cosmetic layer that undergoes a volume change by an external stimulus (provided that, an external stimulus due to contact with a solvent and an external stimulus due to contact with a solution are excluded),
the cover coat layer having a porosity of less than or equal to 40% and a tensile modulus of more than or equal to 5 MPa.

16. The cover coat layer according to claim 15, wherein
the cover coat layer has a transmittance of more than or equal to 10% for light having any wavelength ranging from more than or equal to 280 nm and less than 400 nm.

17. A nail production method comprising:
applying a resin composition for a nail including a stimulus-responsive material that undergoes a volume change by an external stimulus (provided that, an external stimulus due to contact with a solvent and an external stimulus due to contact with a solution are excluded) to a fingernail, and solidifying the resin composition for a nail to form a first nail layer which adheres to the fingernail; and
forming a cover coat layer having a porosity of less than or equal to 40% on the first nail layer.

18. The method according to claim 17, further comprising:
forming a second nail layer on the cover coat layer,
wherein
the second nail layer is a color nail or a top nail.

19. A method of removing a nail which has adhered to a fingernail, the nail including a first nail layer formed of a solidified resin composition for a nail including a stimulus-responsive material that undergoes a volume change by an external stimulus (provided that, an external stimulus due to contact with a solvent and an external stimulus due to contact with a solution are excluded), the method comprising:
forming a cover coat layer having a porosity of less than or equal to 40% on an exposed surface of the first nail layer; and
applying the external stimulus to the first nail layer to remove the first nail layer and the cover coat layer from the fingernail.

20. The method according to claim 19, wherein
the nail further includes at least one other nail layer laminated to the first nail layer, and
the method further comprises removing at least a part of the at least one other nail layer to expose at least a part of the surface of the first nail layer before forming the cover coat layer.

21. A method of removing a nail which has adhered to a fingernail, the nail including a first nail layer formed of a solidified resin composition for a nail including a stimulus-responsive material that undergoes a volume change by an external stimulus (provided that, an external stimulus due to contact with a solvent and an external stimulus due to contact with a solution are excluded), and a cover coat layer disposed on the first nail layer and having a porosity of less than or equal to 40%, the method comprising:
applying the external stimulus to the first nail layer to remove the first nail layer and the cover coat layer from the fingernail.

22. The method according to claim 21, wherein the nail further includes at least one other nail layer laminated to the first nail layer and the cover coat layer, and
the method further comprises removing at least a part of the at least one other nail layer except the first nail layer and the cover coat layer before applying the external stimulus to the first nail layer.
